(19) United States
(12) Patent Application Publication
(10) Pub. No.: US 2026/0240763 A1
(43) Pub. Date: Aug. 20, 2026

(54) Inhalable Serotonin Receptor Agonist Formulations

(71) Applicant: Pinata Holdings Inc., Los Angeles, CA (US)

(72) Inventors: Michael OGBURN, Lake Forest, CA (US); Christopher PRICE, Monroe Township, NJ (US)

(21) Appl. No.: 18/994,166

(22) PCT Filed: Jul. 18, 2023
(86) PCT No.: PCT/US2023/070368

§ 371 Date: Jan. 14, 2025

Related U.S. Application Data
(60) Provisional application No. 63/390,382, filed on Jul. 19, 2022.
(60) Provisional application No. 63/425,401, filed on Nov. 15, 2022.
(60) Provisional application No. 63/432,184, filed on Dec. 13, 2022.
(60) Provisional application No. 63/440,174, filed on Jan. 20, 2023.

(51) Int. Cl. A61K 9/00; A61K 9/50; A61K 31/135; A61K 31/55; A61K 31/675

(52) U.S. Cl. CPC A61K 9/0075; A61K 9/5042; A61K 31/135; A61K 31/55; A61K 31/675

(57) ABSTRACT

Provided herein are pharmaceutical compositions, kits comprising pharmaceutical compositions, methods of treating disease, and methods of making compositions. The pharmaceutical compositions described herein are powdery pharmaceutical compositions, which can contain encapsulated pharmaceutical agents, such as serotonin receptor agonists. The powdery pharmaceutical compositions may be administered by an inhaler device described herein.

### CROSS-REFERENCE

This application claims the benefit of U.S. Provisional Application No. 63/390,382, filed Jul. 19, 2022, of U.S. Provisional Application No. 63/425,401, filed Nov. 15, 2022, of U.S. Provisional Application No. 63/432,184, filed Dec. 13, 2022, and of U.S. Provisional Application No. 63/440,174, filed Jan. 20, 2023, the disclosures of which are incorporated herein by reference in their entirety.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY

Disclosed herein are powdery pharmaceutical compositions, comprising: a) particles of a pharmaceutically acceptable excipient; and b) a plurality of spray dried particles. In some embodiments, each particle of the plurality of spray dried particles can be substantially encapsulated in a coating material. In some embodiments, the plurality of spray dried particles substantially encapsulated in the coating material can comprise: an agonist selected from the group consisting of: a substituted tryptamine or a pharmaceutically acceptable salt thereof, an amide of lysergic acid or a pharmaceutical acceptable salt thereof, and a N-methyl-d-aspartate receptor agonist or a pharmaceutical acceptable salt thereof. In some embodiments, the spray dried particles substantially encapsulated in the coating material individually can have a particle diameter ranging from about 1 micrometer to about 10 micrometers, as measured by a particle analyzer using laser diffraction. In some embodiments, the coating material can comprise a trehalose, a hydroxypropyl methylcellulose (HPMC), a fumaryl diketopiperazine (FDKP), a 1,2-distearoyl-sn-glycero-3-phosphocholine, a hydroxypropyl methylcellulose acetate succinate (HPMCAS), a povidone, a copovidone, or any combination thereof. In some embodiments, the spray dried particles can comprise the substituted tryptamine or the pharmaceutically acceptable salt thereof. In some embodiments, the substituted tryptamine can be a psilocin or a salt thereof. In some embodiments, the substituted tryptamine can be a psilocybin or a salt thereof. In some embodiments, the substituted tryptamine can be an ibogaine or a salt thereof. In some embodiments, the spray dried particles can comprise the N-Methyl-D-aspartate receptor agonist or the pharmaceutically acceptable salt thereof. In some embodiments, the N-Methyl-D-aspartate receptor agonist can be a ketamine or a salt thereof. In some embodiments, the powdery pharmaceutical composition can be for inhaled use or for intranasal use. In some embodiments, the pharmaceutical composition can be in unit dose form. In some embodiments, at least a portion of the particles of the pharmaceutically acceptable excipient individually can have a particle diameter ranging from about 30 micrometers to about 100 micrometers. In some embodiments, the particles of the pharmaceutically acceptable excipient and the plurality of spray dried particles can be admixed into a substantially homologous mixture. In some embodiments, the powdery pharmaceutical composition can be contained within a capsule. In some embodiments, the capsule can be about one quarter to about one half, by volume, filled with the powdery pharmaceutical composition. In some embodiments, a weight-to-weight ratio of: a) the particles of the pharmaceutically acceptable excipient and b) the plurality of spray dried particles, can range from about 1:1 (w/w) to about 10000:1 (w/w). In some embodiments, the weight-to-weight ratio of: a) the particles of the pharmaceutically acceptable excipient and b) the plurality of spray dried particles, can range from about 1:1 (w/w) to about 10:1 (w/w). In some embodiments, the portion of the capsule not containing the powdery pharmaceutical composition can comprise an inert gas. In some embodiments, the capsule can comprise a hydroxypropylmethyl cellulose (HPMC) capsule. In some embodiments, the capsule can be size: 000, 00, 0, 1, 2, 3, or 4. In some embodiments, the capsule can be size 3. In some embodiments, the powdery pharmaceutical composition can be contained within an inhaler unit. In some embodiments, the capsule can be contained in an inhaler unit. In some embodiments, the pharmaceutically acceptable excipient can comprise a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof. In some embodiments, the pharmaceutically acceptable excipient or pharmaceutically acceptable salt thereof can comprise the carbohydrate or the pharmaceutically acceptable salt thereof. In some embodiments, the carbohydrate or the pharmaceutically acceptable salt thereof can comprise a lactose, a microcrystalline cellulose, a cellulose, a mannitol, a sorbitol, a starch, a starch glycolate, a hydroxypropyl methylcellulose, a hydroxypropyl methylcellulose acetate succinate, a cyclodextrin, a maltodextrin, a croscarmellose sodium, a corn starch, a carrageenan, a sorbitol, a maltitol, a glucose, a pharmaceutically acceptable salt of any of these, or any combination thereof. In some embodiments, the pharmaceutically acceptable excipient or the pharmaceutically acceptable salt thereof can comprise a lactose or a pharmaceutically acceptable salt thereof. In some embodiments, the lactose or the pharmaceutically acceptable salt thereof can comprise a lactose, a sieved lactose, a micronized lactose, a spray dried lactose, an at least substantially anhydrous lactose, a monohydrate lactose, a pharmaceutically acceptable salt thereof, or any combination thereof. In some embodiments, the powdery pharmaceutical composition can retain at least about 90% of the agonist after about 6 months when stored in a sealed container at 25° C. and a room atmosphere having about 50 percent relative humidity, as measured by high-performance liquid chromatography (HPLC). In some embodiments, the agonist can be present in an amount ranging from about 0.001 mg to about 20 mg. In some embodiments, the agonist can be in the form of a pharmaceutically acceptable salt thereof and can be a hydrochloride salt, a bitartrate salt or a borate salt. In some embodiments, the particles comprising the agonist can comprise a median diameter of less than 5 μm. In some embodiments, the particles comprising the agonist or the pharmaceutically acceptable salt thereof can comprise a median diameter of less than about: 6 μm, 7 μm, 8 μm, 9 μm or 10 μm.

Also disclosed herein are kits comprising the powdery pharmaceutical composition of disclosed above contained at least in part in a container.

Also disclosed herein are methods of treating a disease or condition in a subject in need thereof, comprising administering by inhalation administration, a first therapeutic comprising a therapeutically effective amount of the powdery pharmaceutical composition disclosed above to the subject in need thereof thereby treating the disease or condition. In some embodiments, the administering can be conducted one, twice, three, or four times per day. In some embodiments, the disease or condition can be selected from the group consisting of: a migraine headache, a tension headache and a cluster headache. In some embodiments, the disease or condition can be selected from the group consisting of: a post-traumatic stress disorder, a depression, a generalized anxiety disorder, a panic disorder, a phobia, a social anxiety disorder, an attention deficit hyperactivity disorder, an agoraphobia and a personality disorder. In some embodiments, the disease or condition can be selected from the group consisting of: an alcohol addiction, a cocaine addiction, a methamphetamine addiction, an opioid addiction, a nicotine addiction, and a gambling addiction. In some embodiments, the powdery pharmaceutical composition can be administered as needed, or for about: one day, two days, three days, four days, five days, six days, a week, two weeks, three weeks, a month, two months, three months, four months, five months, six months, seven months, eight months, nine months, ten months, eleven months, a year, or chronically. In some embodiments, a second therapeutic or pharmaceutically acceptable salt thereof can be administered. In some embodiments, the second therapeutic or a pharmaceutically acceptable salt thereof can be administered concurrently or consecutively. In some embodiments, the second therapeutic or the pharmaceutically acceptable salt thereof can be comprised in the powdery pharmaceutical formulation. In some embodiments, the first therapeutic can comprise the substituted tryptamine or the pharmaceutically acceptable salt thereof and the second therapeutic can comprise the N-Methyl-D-aspartate receptor agonist or the pharmaceutically acceptable salt thereof. In some embodiments, the first therapeutic can comprise the amide of lysergic acid or the pharmaceutically acceptable salt thereof and the second therapeutic can comprise the N-Methyl-D-aspartate receptor agonist or the pharmaceutically acceptable salt thereof. In some embodiments, the method can further comprise administering caffeine, a non-steroidal anti-inflammatory drug, an anti-calcitonin gene-related peptide monoclonal antibody or a combination thereof to the subject. In some embodiments, the subject can be diagnosed with the disease or condition. In some embodiments, the diagnosing can comprise employing an in vitro diagnostic. In some embodiments, the in vitro diagnostic can be a companion diagnostic. In some embodiments, the powdery pharmaceutical composition can be contained within a capsule. In some cases, the capsule can at least in part be contained within an inhaler, and wherein the inhaler contains a sharp surface configured to puncture or slice the capsule, and wherein, prior to administrating, the inhaler is actuated such that the sharp surface punctures or slices the capsule. In some embodiments, the inhalation administration can be oral inhalation, intra nasal administration, or any combination thereof.

Also disclosed herein are powdery pharmaceutical compositions, comprising: a) particles of a pharmaceutically acceptable excipient; and b) a plurality of spray dried particles. In some embodiments, each particle of the plurality of spray dried particles can be substantially encapsulated in a coating material. In some embodiments, the plurality of spray dried particles substantially encapsulated in the coating material can comprise N-lactoyl-phenylalanine or a pharmaceutically acceptable salt thereof. In some embodiments, at least a portion of the spray dried particles substantially encapsulated in the coating material can individually have a particle diameter ranging from about 1 micrometer to about 10 micrometers, as measured by a particle analyzer using laser diffraction. In some embodiments, the coating material can comprise a trehalose, a hydroxypropyl methylcellulose (HPMC), a fumaryl diketopiperazine (FDKP), 1,2-distearoyl-sn-glycero-3-phosphocholine, a hydroxypropyl methylcellulose acetate succinate (HPMCAS), a povidone, a copovidone or any combination thereof. Also disclosed herein are methods of treating a disease or condition in a subject in need thereof, comprising administering by inhalation administration, a composition comprising a therapeutically effective amount of a powdery pharmaceutical composition to the subject in need thereof thereby treating the disease or condition. In some embodiments, the administering can be conducted one, twice, three, or four times per day. In some embodiments, the disease or condition can be selected from the group consisting of: an obesity, an increased appetite, or a glucose intolerance.

### BRIEF DESCRIPTION OF THE DRAWINGS

FIG. 1A shows a dry powder inhaler device for delivery of a powdery pharmaceutical composition to the lung alveolar and FIG. 1B shows a nasal inhaled device for intranasal delivery of a powdery pharmaceutical composition to the lung alveolar.

FIG. 2 shows the method of use for the dry powder inhaler device for delivery of a powdery pharmaceutical composition.

FIG. 3 shows a spray drying manufacturing system comprising a closed spray dryer container which receives a solution comprising a drug dissolved or mixed in a suitable solvent. The system generates solid particles from the solution comprising the drug.

FIG. 4 shows a protective cap for a dry powder inhaler device.

FIG. 5 shows a rotatable mouthpiece of a dry powder inhaler device.

FIG. 6 shows a lower base chamber receptacle of a dry powder inhaler device.

FIG. 7 shows a lateral button operably connected to a sharp surface for use in a dry powder inhaler device for piecing a capsule containing a dry powdery pharmaceutical composition.

FIG. 8 shows a base plate of a dry powder inhaler device.

FIG. 9 shows a dry powder inhaler device for delivery of a powdery pharmaceutical composition to the lung alveolar.

FIG. 10 shows a spray drying manufacturing system comprising a closed spray drying chamber which receives a solution comprising an active ingredient, an encapsulating polymer, and a suitable solvent.

### DETAILED DESCRIPTION

### Overview

Delivering pharmaceutical compositions through oral ingestion of capsules or tablets may take a long time to dissolve and reach the blood stream. The absorption through stomach may take longer if fatty foods are eaten prior to ingestion of the capsule or tablet, further slowing down the process. By spray drying the pharmaceutical compositions and introducing them into the lungs via inhalation, the time needed for the pharmaceutical to reach the blood stream may be significantly reduced. In addition, the dosing level may also be reduced as compared to the oral tablet or capsule equivalent.

Provided herein are methods and pharmaceutical formulations for the treatment of neurological conditions such as headaches and post-traumatic stress disorder. Migraine headaches are a prevalent neurological condition characterized by attacks of headache and associated symptoms, such as nausea, vomiting, photophobia, and/or phonophobia. In US and Western Europe, the overall prevalence of migraine sufferers is 11% of the general population (6% males; 15-18% females). The two most common forms of migraine, migraine without aura and migraine with aura, occur on less than 15 days per month and are referred to as episodic forms of migraine (EM). Another type of headache that may be treated by the pharmaceutical formulations herein are cluster headaches. Cluster headaches are short painful headaches that occur every day for weeks or months and can occur seasonally. Their cause is unknown but involves the trigeminal nerve in the face and pain is felt behind one eye and on one side of the face. Certain other headaches such as a hybrid headache exhibiting symptoms of migraine headaches such as auras but with bi lateral pain or intense debilitating headaches lasting once a day for a few days may be amenable to treatment with the pharmaceutical formulations provided herein.

Provide herein among other things is the production pharmaceutical compositions for the delivery of serotonin agonists for the treatment of a headache and other conditions in a subject. In certain cases, the headache is a migraine headache or a cluster headache. In some instances, a serotonin agonist is a drug that is capable of binding to serotonin receptors. The drug may be delivered as a dry powder drug utilizing inhalation or intranasal administration as the route of administration. In some cases, the drug may be microencapsulated.

Provided herein among other things is the production of pharmaceutical compositions for the delivery of N-Methyl-D-aspartate receptor agonist for the treatment of headaches such as migraine headaches and cluster headaches. In some instances, a N-Methyl-D-aspartate receptor agonist is a drug that is capable of binding to N-Methyl-D-aspartate receptors. The drug may be delivered as a drug powder utilizing inhalation or intranasal administration as the route of administration. In some cases, the drug may be microencapsulated.

Further provided herein are the use of serotonin receptor agonists such as triptans for the treatment of obesity or otherwise to decrease body fat. Treatment may result in: an increase in energy, a decrease in body fat, a cessation in increasing body fat for a duration of time, a reduction in a rate of body fat increase over time, a decrease in appetite, an increase in body flexibility, an increase in posture, an increase in range of movement, a decrease in musculoskeletal pain, or any combination thereof.

Further provided herein are the use of serotonin receptor agonists and N-Methyl-D-aspartate receptor agonists for the treatment of depression and post-traumatic stress disorder. In some instances, N-lactoyl-phenylalanine is provided and delivered as a dry powder utilizing inhalation or intranasal administration as the route of administration. In some cases, the drug may be microencapsulated. In some cases, N-lactoyl-phenylalanine can be administered to treat a weight issue, such as obesity.

Provided herein are pharmaceutical compositions, kits comprising pharmaceutical compositions, methods of treating and preventing disease, and methods of making compositions and kits described herein. Pharmaceutical drugs described herein may be produced employing various methods to synthesize, manipulate, and administer particles. In some aspects, the pharmaceutical compositions described herein are powdery pharmaceutical compositions.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various aspects may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement and include determining if an element may be present or not (for example, detection). These terms may include quantitative, qualitative or quantitative, and qualitative determinations. Assessing may be alternatively relative or absolute. “Detecting the presence of” includes determining the amount of something present, as well as determining whether it may be present or absent.

The terms “subject,”, “host” “individual,” or “patient” are often used interchangeably herein and can refer to animals, typically mammalian animals. In some cases, a “subject” may be a biological entity containing expressed genetic materials. The biological entity may be a plant, animal, or microorganism, including, for example, bacteria, viruses, fungi, and protozoa. The subject may be tissues, cells and their progeny of a biological entity obtained in vivo or cultured in vitro. The subject may be a mammal, such as a human. The subject may be diagnosed or suspected of being at high risk for a disease. In some cases, the subject may not be necessarily diagnosed or suspected of being at high risk for the disease.

The term “substantially” or “essentially” may refer to a qualitative condition that exhibits an entire or nearly total range or degree of a feature or characteristic of interest. In some cases, substantially encapsulated may refer to near complete encapsulation of a substance or compound. For example, substantially encapsulated may comprise a particle that is at least about: 70%, 75%, 80%, 85%, 90%, 95%, 99%, or 100% encapsulated. In some cases, substantially may refer to at least about: 70%, 75%, 80%, 85%, 90%, 95%, 99%, or 100% of the total range or degree of a feature or characteristic of interest.

The term “at least partially” may refer to a qualitative condition that exhibits a partial range or degree of a feature or characteristic of interest. In some cases, at least partially encapsulated may refer to a partial encapsulation of a substance or compound. For example, at least partially encapsulated may comprise a particle that is at least about: 5%, 10%, 20%, 30%, 40%, 50%, 60%, 70%, 75%, 80%, 85%, 90%, 95%, 99%, or 100% encapsulated.

The term “in vivo” may be used to describe an event that takes place in a subject's body.

The term “ex vivo” may be used to describe an event that takes place outside of a subject's body. An “ex vivo” assay may not be performed on a subject. Rather, it may be performed upon a sample separate from a subject. An example of an “ex vivo” assay performed on a sample may be an “in vitro” assay.

The term “in vitro” may be used to describe an event that takes place contained in a container for holding laboratory reagent such that it may be separated from the living biological source organism from which the material may be obtained. In vitro assays may encompass cell-based assays in which cells alive or dead are employed. In vitro assays may also encompass a cell-free assay in which no intact cells are employed.

As used herein, the term ‘about’ a number may refer to that number plus or minus 10% of that number. The term ‘about’ a range may refer to that range minus 10% of its lowest value and plus 10% of its greatest value.

As used herein, a “derivative” of a compound disclosed herein, can refer to a chemical substance related structurally a compound disclosed herein. A derivative can be made from the structurally-related parent compound in one or more steps. The general physical and chemical properties of a derivative can be similar to a parent compound. A derivative can be, for example, an analog or a homolog.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit may be achieved with the eradication or amelioration of one or more of the physiological symptoms associated with the underlying disorder such that an improvement may be observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the term “unit dose” or “dosage form” may be used interchangeably and may be meant to refer to pharmaceutical drug products in the form in which they are marketed for use, with a specific mixture of active ingredients and inactive components or excipients, in a particular configuration, and apportioned into a particular dose to be delivered. The term “unit dose” may also sometimes encompass non-reusable packaging, although the FDA distinguishes between unit dose “packaging” or “dispensing”. More than one unit dose may refer to distinct pharmaceutical drug products packaged together, or to a single pharmaceutical drug product containing multiple drugs and/or doses. The term “unit dose” may also sometimes refer to the particles comprising a pharmaceutical composition, and to any mixtures involved. Types of unit doses may vary with the route of administration for drug delivery, and the substance(s) being delivered. A solid unit dose may be the solid form of a dose of a chemical compound used as a pharmaceutically acceptable drug or medication intended for administration or consumption.

As used herein, the term “fine particle fraction” or “fine particle fraction from the emitted dose” may refer to the mass of active agent having an aerodynamic diameter below about: 5 μm, 6 μm, 7 μm, 8 μm, 9 μm, or 10 μm. In some instances, the cutoff size may be less than or equal to an aerodynamic diameter of about 5 μm. In some instances, the cutoff size may be less than or equal to an aerodynamic diameter of about 6.4 μm. In some instances, the cutoff size may be less than or equal to an aerodynamic diameter of about 7 μm or about 8 μm. In some instances, the fine particle fraction may be often used to evaluate the efficiency of aerosol deaggregation. In some cases, fine particle fraction may be the mass of active agent having an aerodynamic diameter below about: 5 μm, 6 μm, 7 μm, 8 μm, 9 μm, or 10 μm as a percentage of an emitted dose mass. In some cases, fine particle fraction may be the mass of active agent having an aerodynamic diameter of more than about: 5 μm, 6 μm, 7 μm, 8 μm, 9 μm, or 10 μm as a percentage of an emitted dose mass. In some cases, fine particle fraction may be the mass of active agent having an aerodynamic diameter from about: 1 μm to about 10 μm, 1 μm to about 5 μm, 3 μm to about 6 μm, 4 μm to about 7 μm, 6 μm to about 12 μm or about 7 μm to about 10 μm as a percentage of an emitted dose mass. For example, a composition described herein may have a fine particle fraction of more than, less than, or equal to about: 20%, 25%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 75%, 80%, 85%, 90% or 95% upon aerosolization.

As used herein, a “dose” may refer to a measured quantity of a therapeutic agent to be taken at one time.

As used herein, “pharmaceutically acceptable salt” may refer to pharmaceutical drug molecules, which may be formed as a weak acid or base, chemically made into their salt forms, most frequently as the hydrochloride, sodium, or sulfate salts. Drug products synthesized as salts may enhance drug dissolution, boost absorption into the bloodstream, facilitate therapeutic effects, and increase its effectiveness. Pharmaceutically acceptable salts may also facilitate the development of controlled-release dosage forms, improve drug stability, extend shelf life, enhance targeted drug delivery, and improve drug effectiveness.

As used herein, “laser diffraction” may refer to a method for particle size analysis, which consists of scattering laser light off an assembly of particles, and collecting the scattered light using a spatial array of detectors. The signal from the detectors may be a pattern of scattered/diffracted light vs. angle. This pattern may result from many particles being illuminated by the laser light source at the same time, where all of their individual scattered/diffracted light rays mix together at each detector element.

As used herein, “particle size analyzer” may refer to an instrument for particle size analysis, particle size measurement, or simply particle sizing.

As used herein, “particle size analysis” may refer to the collective name of the technical procedures, or laboratory techniques which determines the size range, and/or the average (mean), median or mode size of the particles, or droplets in a powder or liquid sample.

As used herein, “time to peak plasma concentration” may refer to the time required for a drug to reach peak concentration in plasma. Peak concentration in plasma may be usually defined as the plasma concentration that a drug achieves in a specified compartment or test area of the body after the drug has been administered and before the administration of a second dose.

As used herein, “HPLC” may refer to high-performance liquid chromatography (formerly referred to as high-pressure liquid chromatography), which is a technique in analytical chemistry used to separate, identify, and quantify each component in a mixture. HPLC may be a common technique used in pharmaceutical development, as it may be a method to ensure product purity.

As used herein, the terms “effective amount” or “therapeutically effective amount” of a drug used to treat a disease may be an amount that may reduce the severity of a disease, reduce the severity of one or more symptoms associated with the disease or its treatment, or delay the onset of more serious symptoms or a more serious disease that may occur with some frequency following the treated condition. An “effective amount” may be determined empirically and in a routine manner, in relation to the stated purpose.

The section headings used herein are for organizational purposes only and are not to be construed as limiting the subject matter described.

### Pharmaceutical Formulations

Disclosed herein are compositions, such as pharmaceutical compositions comprising a substituted tryptamine, an amide of lysergic acid, a N-methyl-d-aspartate receptor agonist, a serotonin receptor agonist, a N-lactoyl-phenylalanine, a salt of any of these, or any combination thereof. Devices, systems and methods for producing, packaging, and delivering stable powdery pharmaceutical compositions are also disclosed herein. The active ingredient of these stable powdery formulations may comprise serotonin receptor agonists, a N-Methyl-D-aspartate receptor agonist or both. In some cases, the active ingredient may comprise a serotonin receptor agonist of the triptan class, a N-lactoyl-phenylalanine, or both. The active ingredients may further comprise additional ingredients as disclosed below. In certain instances, the dry powdery compositions may include inactive ingredients, such as excipients, carriers and/or diluents.

In certain instances, the dry powdery pharmaceutical compositions can be inhalable. For example, the dry powdery pharmaceutical compositions can be administered in a dry powdered inhaler. In other instances, the dry powdery pharmaceutical compositions can be for oral administration. For example, the dry powdery pharmaceutical compositions can be administered in a capsule-in capsule formulation. In another example, the dry powdery pharmaceutical compositions can be administered as microencapsulated particles with multiple layers.

### Active Ingredients and Pharmaceutically Acceptable Salts

An active pharmaceutical ingredient may be any substance or mixture of substances intended to be used in the manufacture of a drug (medicinal) product and that, when used in the production of a drug, becomes an active ingredient of the drug product. Such substances may be intended to furnish pharmacological activity or other direct effect in the diagnosis, cure, mitigation, or treatment of disease or to affect the structure or function of the body. In some aspects, an active pharmaceutical ingredient or salt thereof may be formulated as a powder. For example, inhalable serotonin receptor agonist formulations disclosed herein may be formulated as a powder using the methods described herein. In some cases, an active ingredient comprises a pharmaceutical compound. In some cases, a pharmaceutical compound comprises an active ingredient.

In some aspects, the active pharmaceutical ingredients may comprise serotonin receptor agonists or pharmaceutically acceptable salts thereof. In some aspects, the serotonin receptor agonist can be an agonist for serotonin receptor 5-HT_{1A}, 5-HT_{1B}, 5-HT_{1D}, 5-HT_{1E}, 5-HT_{1E}, 5-HT_{1F}, 5-HT_{2A}, 5-HT_{2A}, 5-HT_{2B}, 5-HT_{2C}, 5-H_{T3}, 5-H_{T4}, 5-HT_{5A}, 5-HT_{5B}, 5-H_{T6}, 5-H_{T7}, or a combination of these. In certain cases, the serotonin receptor agonist is a substituted tryptamine such as: 4-phosphoryloxy-N,N-dimethyltryptamine (psilocybin), 4-hydroxy-N,N-dimethyltryptamine (psilocin), 4-hydroxyindole-3-acetaldehyde, 4-hydroxytryptophol, 4-hydroxyindole-3-acetic acid, a combination of these, a derivative of these, or a salt of these. In certain instances the substituted tryptamine may be a synthetic analog of psilocin such as: 1-Methylpsilocin, 4-Fluoro-N,N-dimethyltryptamine, 4-Acetoxy-N,N-dimethyltryptamine (O-Acetylpsilocin), 4-hydroxy-N-methyl-N-isopropyltryptamine, 4-hydroxy-N-methyltryptamine (norpsilocin), 4-Acetoxy-N-methyl-N-ethyltryptamine (4-Acetoxy-MET), 4-acetoxy-N-methyl-N-isopropyltryptamine (4-Acetoxy-MiPT), 4-acetoxy-N,N-diisopropyltryptamine (4-Acetoxy-DiPT), 4-hydroxy-N-methyl-N-ethyltryptamine (4-HO-MET), 4-hydroxy-N-isopropyl-N-methyltryptamine (4-HO-MiPT), 4-Hydroxy-N-methyl-N-propyltryptamine (4-HO-MPT), 4-Hydroxy-N, N-diisopropyltryptamine (4-HO-DiPT), 4-Hydroxy-5-methoxy dimethyltryptamine (4-HO-5-MeO-DMT), a combination of these, a derivative of these, or a salt of these. In some cases, an active pharmaceutical ingredient can comprise 4-AoC-DMT (O-Acetylpsilocin), a salt thereof, or a derivative thereof.

In some cases, an active pharmaceutical ingredient can be synthetically manufactured or purified from an animal and/or plant. For example, a serotonin receptor agonist can be synthetically produced. In another example, a psilocybin or a salt thereof can be a synthetic psilocybin or a salt thereof, a psilocin or a salt thereof can be a synthetic psilocin or a salt thereof, and an ibogaine or a salt thereof can be a synthetic ibogaine or a salt thereof. In some cases, a N-Methyl-D-aspartate receptor agonist can be a synthetic N-Methyl-D-aspartate receptor agonist. In some cases, an amide of lysergic acid can be a synthetic amide of lysergic acid. In some cases, In some cases, a 4-AoC-DMT can be a synthetic 4-AoC-DMT.

Additional substituted tryptamines that have been synthetically manufactured and can be included in a composition include: N,N-diethyltryptamine (DET), N-Ethyl-N-propyltryptamine (EPT), 4-propionoxy-N,N-dimethyltryptamine (4-PrO-DMT), 4-Acetoxy-N-methyl-N-ethyltryptamine (4-Acetoxy-MET), ethylisopropyltryptamine (EiPT), N-methyl-N-butyltryptamine (MBT), propylisopropyltryptamine (PiPT), N,N-Dipropyltryptamine (DPT), N,N-diisopropyltryptamine (DiPT), N,N-Diallyltryptamine (DALT), N,N-Dibutyltryptamine (DBT), N-Ethyltryptamine (NET), 2-N,N-trimethyltryptamine (2-Me-DMT), 5-N,N-trimethyltryptamine (5-TMT), 7-N,N-trimethyltryptamine (7-TMT), α-Methyltryptamine (AMT), a-Ethyltryptamine (AET), α-N-dimethyltryptamine (Alpha, N-DMT), α-N,N-Trimethyltryptamine (ATMT), 4-hydroxy-diethyl-tryptamine (4-HO-DET), 4-Hydroxy-N,N-dibutyltryptamine (4-HO-DBT), 4-hydroxy-N,N-di-sec-butyltryptamine (4-HO-DsBT), 4-Hydroxy-a-methyltryptamine (4-13-HO-αMT), 4-Hydroxy-N-methyl-(α,N-trimethylene)-tryptamine (4-HO-MPMI), 4-Acetoxy-N,N-diethyltryptamine (4-AcO-DET), 4-Acetyloxy-N,N-dipropyltryptamine (4-AcO-DPT), 4-acetoxy-N, N-diisopropyltryptamine (4-AcO-DiPT), 4-Acetyloxy-N,N-diallyltryptamine (4-AcO-DALT), 4-methoxy-N,N-dimethyltryptamine (4-MeO-DMT), 4-methoxy-N-methyl-N-isopropyltryptamine (4-MeO-MiPT), 5-Methoxy-N-methyl-N-ethyltryptamine (5-MeO-MET), 5-Methoxy-N-methyl-N-isopropyltryptamine (5-MeO-MiPT), 5-methoxy-N-methyl-N-allyltryptamine (5-MeO-MALT), 5-methoxy-N,N-diethyltryptamine (5-MeO-DET), 5-methoxy-N-ethyl-N-Isopropyltryptamine (5-MeO EiPT), 5-methoxy-N-ethyl-N-propyltryptamine (5-methoxy EPT), 5-methoxy-N, N-dipropyltryptamine (5-MeO-DPT), 5-Methoxy-N,N-diisopropyltryptamine (5-MeO-DiPT), N,N-di allyl-5-methoxy tryptamine (5-MeO-DALT), 5-Methoxy-alpha-ethyltryptamine (5-MeO-a-ET), 5-Methoxy-N-methyl-(α,N-trimethylene)tryptamine) (5-MeO-MPMI), 5-Methoxy-2,N, N-trimethyltryptamine (5-MeO-TMT), 5-Methoxy-7,N,N-trimethyltryptamine (5-MeO-7-TMT), α,N-dimethyl-5-methoxytryptamine (5-MeO-a, N-DMT), 4-Fluoro-5-Methoxy-N,N-dimethyltryptamine(4-F-5-MeO-DMT), 5-methylthio-N,N-dimethyltryptamine (5-MeS-DMT), 5,N-Dimethyl-N-isopropyltryptamine (5-Me-MiPT), 5hydroxy-N,N-di-iso-propyltryptamine (5-HO-DiPT), 2,α-Dimethyltryptamine (2,α-DMT), a combination of these, a derivative of any of these, or a salt of any of these.

Additional substituted tryptamines that may be used in a composition herein include: 5-hydroxy-N-methyltryptamine (norbufotenin), 5-hydroxy-N,N-dimethyltryptamine (bufotenin), 3-[2-(trimethylazaniumyl)ethyl]-1H-indol-5-olate (bufotenidine), 5-methoxy-N-acetyltryptamine (melatonin), 4-phosphoryloxy-tryptamine (norbaeocystin), 4-phosphoryloxy-N-methyl-tryptamine (baeocystin), [3-[2-(trimethylazaniumyl)ethyl]-1H-indol-4-yl] hydrogen phosphate (aeruginascin), N,N-dimethyltryptamine (DMT), 5-methoxy-N, N-dimethyltryptamine (5-MeO-DMT), 5-bromo-N,N-dimethyltryptamine (5-Bromo-DMT), 4-phosphoryloxy-N,N-diethyltryptamine (ethocybin), N-Methyltryptamine (NMT), a combination of any of these, a derivative of any of these, or a salt of any of these.

In certain instances, a substituted tryptamine may be a triptan such as a almotriptan, a rizatriptan, a sumatriptan, a zolmitriptan, an eletriptan, a naratriptan, a donitriptan, a frovatriptan, a LY-334370, a L-694247, a combination of any of these, a derivative of any of these, or a salt of any of these. In some cases, a triptan can be used in a composition. In certain cases, the triptan, derivative thereof, or salt thereof may be microencapsulated.

In certain cases, the serotonin receptor agonist can be used in a composition herein. In certain cases, the serotonin receptor agonist is an amide of lysergic acid such as an ergine, an ergometrine, an ergotamine, a methylergometrine, a methysergide, an amesergide, a lysergic acid diethylamide, a 1-acetyl-LSD, a combination of any these, a derivative of any of these, or a salt of any of these.

In some cases, a pharmaceutical compound may comprise a small molecule agonist which binds to N-Methyl-D-aspartate receptor agonist such as a ketamine, a dextromethorphan, a phencyclidine, a methoxetamine or a combination of any of these, a derivative of any of these or a pharmaceutically acceptable salt of any of these.

In certain instances, the tryptamine may be in the form of an ayahuasca or a salt thereof. In certain instances, the ayahuasca may be made from a plant material such as from Banisteriopsis caapi, Psychotria viridis, Justicia pectoralis, Brugmansia insignis, Brugmansia versicolor, Nicotiana rustica, Datura strmonium, Datura wrightii, a combination of these, a subspecies of these, an isolate of these, or a combination thereof. In certain instances, ayahuasca may comprise a tryptamine such as a DMT or a salt thereof, which itself may be derived from, for example, Banisteriopsis caapi, and an anticholinergic deliriant or a salt thereof such as one derived from Datura wrightii.

In some instances, a therapeutic or pharmaceutically acceptable salt thereof may be an tryptamine such as a mitragynine, a 7-hydroxymitragynine (7-HMG), a raubasine, a mitraphylline, a mitragynine pseudoindoxyl, a rhynchophylline, an ibogaine, such as ibogane derived from plants in the family Apocynaceae such as Tabernanthe iboga, Voacanga africana, and Tabernaemontana undulata, a voacangine, an ibogamine, a noribogaine, 18-methoxycoronaridine, an alkaloid derived from kratom, a combination of any of these, a derivative of any of these, or a pharmaceutically acceptable salt of any of these.

In certain instances, a tryptamine may be an agonist for additional receptors. Additional receptors may include: a μ-opiod receptor, a κ-opiod receptor, a nicotinic acetylcholine receptor (nAChR α3β4 and α2β4), a sigma-1 receptor (σ1), a sigma-2 receptor (σ2), a trace amine-associated receptor (TAARs), or a combination of any these.

In certain cases, the active pharmaceutical ingredients described herein (e.g., an agonist) or their pharmaceutically acceptable salts thereof can be used to treat a post-traumatic stress disorder, a depression, an anxiety, a generalized anxiety disorder, a panic disorder, a phobia, a social anxiety disorder, an attention deficit hyperactivity disorder, an agoraphobia, a personality disorder, or any combination thereof. In some cases, the active pharmaceutical agents act through a serotonin agonist pathway as described above. In certain cases, the active pharmaceutical ingredients described herein or their salts can be used to treat a migraine headache, a tension headache, a cluster headache, a hypnic headache, a sinus headache, or any combination thereof. Additional serotonin agonists that may be employed as those above can include: a mescaline, a methylene-deoxy-methamphetamine (MDMA), derivatives thereof, pharmaceutical salts thereof, or any combination thereof.

In certain further cases, a molecule called N-lactoyl-phenylalanine which floods the body during and immediately after exercise, may be included in a pharmaceutical formulation described herein. Pharmaceutical compositions provided herein may pertain to a pharmaceutical formulation comprising a N-lactoyl-phenylalanine, a derivative thereof, or a pharmaceutical salt thereof that reduces appetite, reduces body fat, and improves glucose tolerance. In some cases, N-lactoyl-phenylalanine or a salt thereof can be administered by a dry powdered inhaler. In some cases, N-lactoyl-phenylalanine or a salt thereof can be administered orally by a capsule or by a capsule in capsule. A capsule may release the pharmaceutical composition for intranasal or inhalation administration.

In certain cases, pharmaceutical compositions provided herein may pertain to a pharmaceutical composition comprising a triptan, a derivative thereof, or a pharmaceutical salt thereof, that reduces body fat. In some cases, the triptan or salt thereof can be administered orally or by a capsule or by a capsule in capsule. A capsule may release the pharmaceutical composition for intranasal or inhalation administration.

In some aspects, the composition may further comprise: another set of active pharmaceutical ingredients or salts thereof. For example, a second, third, or fourth different set of active pharmaceutical ingredients. In some aspects, the additional pharmaceutical ingredients or salts thereof may be administered in parallel or consecutively to enhance the efficacy of the first set of active pharmaceutical ingredients or salts.

In some aspects, a composition may further comprise: an additional set of active pharmaceutical ingredients or salts thereof which may be administered in parallel or consecutively to enhance the efficacy of a serotonin receptor agonist. In some aspects, a composition may further comprise: an additional set of active pharmaceutical ingredients or salts thereof which may be administered in concurrently or consecutively to enhance the efficacy of a serotonin receptor agonist. In some aspects, a second different set of active pharmaceutical ingredients or salts may be administered in parallel or consecutively to enhance the efficacy of a serotonin receptor agonist. In some aspects, a composition may comprise two or more different sets of active pharmaceutical ingredients or salts thereof which may be administered in parallel or consecutively to enhance the serotonin receptor agonist.

In some aspects the first set of active pharmaceutical ingredients or salts may be administered in parallel or consecutively with a second different set of active pharmaceutical ingredients. In some cases, the pharmaceutical ingredients may comprise a nitrate, a nitric oxide, nitric oxide generating components, a nitrite salt, a nitrate salt, a sodium nitrate, a potassium nitrate, a vitamin C, an ascorbic acid, a L-arginine, a L-citrulline, a vitamin B12, a magnesium ascorbate, a sodium ascorbate, a potassium ascorbate, an antihypertensive agent, a diuretic, a salt of any of these, or any combination thereof.

In some aspects, active pharmaceutical ingredients or salts may comprise an antibiotic, an antiviral, an antiparasitic, a diuretic, a blood pressure medication, a phosphodiesterase inhibitor, a pharmaceutically acceptable salt of any of these, or any combination thereof. In some cases, an active pharmaceutical ingredient may comprise a beta-blocker, an ACE inhibitor, an angiotensin II receptor blocker, a calcium channel blocker, an alpha blocker, a cancer chemotherapeutic, a steroid, an immunomodulator, a pharmaceutically acceptable salt of any of these, or any combination thereof. In some cases, an antibiotic may comprise a penicillin, a cephalosporin, a tetracycline, an aminoglycoside, a macrolide, clindamycin, a sulfonamide, a trimethoprim, a metronidazole, a quinolone, or a nitrofurantoin. An antiviral may comprise an acyclovir, peramivir, zanamivir, oseltamivir phosphate, remdesivir, balozavir marboxil, a salt of any of these or any combination thereof. In some cases, an active pharmaceutical ingredient or salt thereof may comprise a potassium channel blocker such as dalfampridine or a salt thereof. In some cases, an active pharmaceutical ingredient or salt thereof may comprise a levodopa, a carbidopa, or a salt thereof.

In some aspects, active pharmaceutical ingredients or salts thereof may comprise a cannabinoid such as a tetrahydrocannabinol, a cannabidiol, a pharmaceutically acceptable salt of any of these, or any combination thereof.

In some cases, the pharmaceutical ingredients may comprise beta blockers (β-blockers), calcium blockers, angiotensin converting enzyme inhibitors, angiotensin receptor blockers, Nebivolol, CYP3A4 inhibitors, ketoconazole (Nizoral), itraconazole (Sporanox), erythromycin, saquinavir, clarithromycin, HIV protease inhibitors, alpha-adrenergic blocking agents (α-blockers), salts thereof, or any combination thereof.

In some aspects, a second different set of active pharmaceutical ingredients or salts as described above may not be comprised in the powdery pharmaceutical composition. In some aspects, a second different set of active pharmaceutical ingredients or salts not comprised in the powdery pharmaceutical composition may be administered concurrently, in parallel, or consecutively. Examples of additional active pharmaceutical ingredients that may be administered concurrently, in parallel, or consecutively include an antibody such as an anti-calcitonin gene-related peptide monoclonal antibody such as eptinezumab, fremanezumab, erenumab, galcanezumab, a derivative of these or a combination of these.

In some aspects, the pharmaceutical composition can have metabolites that may be pharmacologically active, retaining, at least partially, the potency of the parent drug or the parent pharmaceutical component.

In some aspects, the pharmaceutical composition comprises the salt of the pharmaceutically active ingredient. In some cases, the salt can comprise an organic salt, an inorganic salt, or any combination thereof. In some cases, an organic salt may comprise a phosphinate (e.g., sodium hypophosphite), a hydrazinium salt, a urate, a diazonium salt, an oxalate salt, a tartrate, a choline chloride. An example of an inorganic salt may be sodium chloride, calcium chloride, magnesium chloride, sodium bicarbonate, potassium chloride, sodium sulfate, calcium carbonate, calcium phosphate, or any combination thereof.

In some aspects, the pharmaceutical composition comprises the salt of the pharmaceutically active ingredient. In some cases, the salt comprises an HCl salt, an ascorbic acid salt, a mandelic acid salt, an aspartic acid salt, a carbonic acid salt, a citric acid salt, a formic acid salt, a glutamic acid salt, a lactic acid salt, a lauric acid salt, a maleic acid salt, a borate salt, a bitartrate salt, a palmitic acid salt, a phosphoric acid salt, or any combination thereof.

In some aspects, pharmaceutically acceptable salts include, but are not limited to, metal salts such as a sodium salt, a potassium salt, a cesium salt and the like; an alkaline earth metal salt such as a calcium salt, a magnesium salt and the like; an organic amine salts such as a triethylamine salt, a pyridine salt, a picoline salt, a ethanolamine salt, a triethanolamine salt, a dicyclohexylamine salt, a N,N′-dibenzylethylenediamine salt and the like; an inorganic acid salt such as a hydrochloride, a hydrobromide, a phosphate, a sulphate and the like; an organic acid salt such as a citrate, a lactate, a tartrate, a maleate, a fumarate, a mandelate, an acetate, a dichloroacetate, a trifluoroacetate, an oxalate, a formate and the like; a sulfonate such as a methanesulfonate, a benzenesulfonate, a p-toluenesulfonate and the like; and an amino acid salt such as an arginate, an asparginate, a glutamate and the like.

In some aspects, a pharmaceutical composition can comprise a plant, a fungus, a portion thereof, or an extract thereof. In some aspects, a pharmaceutical composition can comprise a at least a portion of a plant, a fungus, or both. In some cases, a plant, a fungus, a portion thereof, or an extract thereof can comprise a Cannabis, a Salvia divorum, a Catha edulis, a Piper methysticum, a Myristica fragrans, a Solanaceae, a Lophophora williamsii, a Lophophora, an Echinopsis peruviana, an Echinopsis pachanoi, an Echinopsis, a Mimosa hostilis, a Chacrunas anadenanthera, a colubrina, an Anadenanthera peregrina, a Mucuna pruriens, an Argyreia nervosa, a Tabernanthe iboga, an Ephedra, an Acacia, a Turnera diffusa, a Calea zacatechichia, a Silene capensisa, a Valeriana officinalis, a Kratom, a Mitragyna speciosa, a Rauvolfia serpentina, a Corynanthe johimbe, a Sceletium tortuosum, a Glaucium flavum, an Eschscholzia californica, or any combination thereof. In some cases, a plant, a fungus, a portion thereof, or an extract thereof can comprise a Psilocybe, a Panaeolus, a Copelandia, an Inocybe, a Pluteus, a Gymnopilus, a Pholiotina, an Amanita muscaria, a Dictyonema huaorani, a Collybia maculata, or any combination thereof. In some cases, a plant, a fungus, a portion thereof, or an extract thereof can be in the form of an active oil extract, a powder (e.g., a ground mushroom, or plant), or both. In some cases, a pharmaceutical composition that comprises a plant, a fungus, a portion thereof, or an extract thereof can be administered in the form of a capsule, or a capsule in capsule formulation. In some cases, a pharmaceutical composition can comprise a flowing agent. In some instances, a capsule, or a capsule in capsule formulation can comprise an enteric coating. In some cases, the pharmaceutical composition comprising a plant, a fungus, a portion thereof, or an extract thereof can be formulated with an: an excipient, a diluent, or a carrier. In some cases, an excipient, a diluent or a carrier can comprise an oat bran filler, a magnesium stearate, a calcium silicate, or any combination thereof. In some instances, a portion of a plant can comprise a flower, a leaf, a root, a stem, a nut, a seed, a fruit, or any combination thereof. In some cases, a portion of a fungus can comprise a cap, a spore, a gill, a ring, a stem, a stalk, a hyphae, a mycelium, a fruiting body, a volva, a mushroom, or any combination thereof.

In some aspects, a pharmaceutical composition can comprise an animal, an extract thereof, or a portion thereof. In some aspects, a pharmaceutical composition can comprise at least a portion of an animal. For example, a pharmaceutical composition can comprise a secretion of an animal. In some cases, an animal, an extract thereof, or a portion thereof can comprise an Incilius alvarius, a Bufo gargarizans, an Osteocephalus taurinus, an Osteocephalus oophagus, an Osteocephalus langsdorfii, a Phyllomedusa, a Phyllomedusa bicolor, a Sarpa salpa, a Siganus spinus, a Kyphosus, a Mulloidichthys flavolineatus, a sea sponge, a Smenospongia aurea, a Smenospongia echina, a Verongula rigida, an Eudistoma fragum, a Paramuricea clavata, a Villogorgia rubra, or any combination thereof. In some cases, an animal, a portion thereof, or an extract thereof can be in the form of an active oil extract, a powder, or both. In some cases, a pharmaceutical composition that comprises an animal, an extract thereof, or a portion thereof can be administered in the form of a capsule, or a capsule in capsule formulation. In some cases, the pharmaceutical composition comprising an animal, an extract thereof, or a portion thereof can be formulated with an: excipient, a diluent, or a carrier. In some cases, an excipient, a diluent or a carrier can comprise an oat bran filler, a magnesium stearate, a calcium silicate, or any combination thereof. In some cases, a pharmaceutical composition can comprise a flowing agent. In some instances, the capsule, or capsule in capsule formulation can comprise an enteric coating. In some cases, an enteric coating can be configured to release the composition in the small or large intestine.

In some cases, a plant, a fungus, an animal, a portion thereof, or an extract thereof can be in a composition in an amount of more than, less than, or equal to about: 0.01 g, 0.05 g, 0.1 g, 0.2 g, 0.3 g, 0.4 g, 0.5 g, 0.6 g, 0.7 g, 0.8 g, 0.9 g, 1 g, 2 g, 3 g, 4 g, 5 g, 6 g, 7 g, 8 g, 9 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, 30 g, 31 g, 32 g, 33 g, 34 g, 35 g, 36 g, 37 g, 38 g, 39 g, 40 g, 41 g, 42 g, 43 g, 44 g, 45 g, 46 g, 47 g, 48 g, 49 g, 50 g, 55 g, 60 g, 65 g, 70 g, 75 g, 80 g, 85 g, 90 g, 95 g, 100 g, 105 g, 110 g, 115 g, 120 g, 125 g, 130 g, 135 g, 140 g, 145 g, 150 g, 155 g, 160 g, 165 g, 170 g, 175 g, 180 g, 185 g, 190 g, 195 g, 200 g, 250 g, 300 g, 350 g, 400 g, 450 g, 500 g, or 1000 g. In some cases, a plant, a fungus, an animal, a portion thereof, or an extract thereof can be in a composition in an amount of about: 0.01 g to about 1000 g, 0,01 g to about 1 g, 0.1 g to about 5 g, 1 g to about 100 grams, 1 gram to about 10 grams, 5 grams to about 20 grams, 10 grams to about 40 grams, 20 grams to about 70 grams, 30 grams to about 90 grams, 40 grams to about 150 grams, 80 grams to about 200 grams, 100 grams to about 500 grams, 250 grams to about 750 grams, or about 500 gram to about 1000 grams. In some cases, the active ingredient in a plant, a fungus, an animal, a portion thereof, or an extract thereof is standardized, such that the same amount or substantially the same amount of the active ingredient is present in each dose of a composition comprising a plant, a fungus, an animal, a portion thereof, or an extract thereof.

### Excipients and Other Non-Active Ingredients

In some aspects, a pharmaceutical composition can comprise a pharmaceutically acceptable: excipient, diluent, and/or carrier. In some cases, a pharmaceutical composition can comprise a flowing agent. As used herein, “excipient” may refer to a substance formulated alongside the active ingredient of a medication, included for the purpose of long-term stabilization, bulking up solid formulations that contain potent active ingredients in small amounts, and/or to confer a therapeutic enhancement on the active ingredient(s) in the final dosage form. In some cases, a diluent can comprise water, or a saline. In some cases, a carrier can comprise a water, a sugar solution, a honey, or a saline. Excipients may facilitate drug absorption, reduce viscosity, or enhance solubility. Excipients may also facilitate the handling of the active ingredients, improve in vitro stability, and/or extend pharmaceutical product shelf life. Excipient selection may vary with the route of administration for drug delivery, the unit dose, as well as the active ingredients comprising the composition.

In some aspects, an excipient may comprise anhydrous calcium phosphate, dihydrate calcium phosphate, hydroxypropyl methylcellulose, croscarmellose sodium, GMO-free croscarmellose sodium, carbomers, magnesium aluminometasilicate, mannitol, povidone (PVP), crospovidone, sorbitol, dimethicone, sodium stearyl fumarate, sodium starch glycollate, hydroxypropylcellulose, native corn starch, modified corn starch, carrageenan, alginates, silicon dioxide, microcrystalline cellulose, carboxymethylcellulose sodium, alginates, carboxymethylcellulose (CMC), sodium carboxymethylcellulose (Na CMC), carbomers, natural gums, sorbitol, maltitol, glucose syrup, silicones, carbomers, fatty alcohols, alcohols, carbohydrates, petrolatum derivatives, butters, waxes, DMSO, esters, fatty acids, oil-in-water (O/W) emulsifiers, water-in-oil (W/O) emulsifiers, silicas, fumed silicas, polysorbates, isopropyl myristate, cellulosic derivates, xanthan gum, propylenglycol, noveon AA-1 polycarbophyl, dimethyl isosorbate, polysilicone elastomer 1100, polysilicone elastomer 1148P, preservatives, flavors, colors, functional coatings, aesthetic coatings, a pharmaceutically acceptable salt of any of these, or any combination thereof.

In some cases, a pharmaceutically acceptable excipient may comprise acacia, acesulfame potassium, acetic acid, glacial, acetone, acetyl tributyl citrate, acetyl triethyl citrate, agar, albumin, alcohol, alginic acid, aliphatic polyesters, alitame, almond oil, alpha tocopherol, aluminum hydroxide adjuvant, aluminum oxide, aluminum phosphate adjuvant, aluminum stearate, ammonia solution, ammonium alginate, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylparaben, calcium alginate, calcium carbonate, calcium phosphate, dibasic anhydrous, calcium phosphate, dibasic dihydrate, calcium phosphate, tribasic, calcium stearate, calcium sulfate, canola oil, carbomer, carbon dioxide, carboxymethylcellulose calcium, carboxymethylcellulose sodium, carrageenan, castor oil, castor oil, hydrogenated, cellulose (e.g., microcrystalline, powdered, silicified microcrystalline, acetate, acetate phthalate) ceratonia, cetostearyl alcohol, cetrimide, cetyl alcohol, cetylpyridinium chloride, chitosan, chlorhexidine, chlorobutanol, chlorocresol, chlorodifluoroethane, chlorofluorocarbons, chloroxylenol, cholesterol, citric acid monohydrate, colloidal silicon dioxide, coloring agents, copovidone, corn oil, cottonseed oil, cresol, croscarmellose sodium, crospovidone, cyclodextrins, cyclomethicone, denatonium benzoate, dextrates, dextrin, dextrose, dibutyl phthalate, dibutyl sebacate, diethanolamine, diethyl phthalate, difluoroethane, dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol palmitostearate, ethylene vinyl acetate, ethylparaben, fructose, fumaric acid, gelatin, glucose, glycerin, glyceryl behenate, glyceryl monooleate, glyceryl monostearate, glyceryl palmitostearate, glycofurol, guar gum, hectorite, heptafluoropropane, hexetidine, hydrocarbons, hydrochloric acid, hydroxyethyl cellulose, hydroxyethylmethyl cellulose, hydroxypropyl cellulose, hydroxypropyl cellulose, low-substituted, hydroxypropyl starch, hypromellose, hypromellose acetate succinate, hypromellose phthalate, honey, imidurea, inulin, iron oxides, isomalt, isopropyl alcohol, isopropyl myristate, isopropyl palmitate, kaolin, lactic acid, lactitol, lactose, anhydrous, lactose, monohydrate, lactose, spray-dried, lanolin, lanolin alcohols, lanolin, hydrous, lauric acid, lecithin, leucine, linoleic acid, macrogol hydroxystearate, magnesium aluminum silicate, magnesium carbonate, magnesium oxide, magnesium silicate, magnesium stearate, magnesium trisilicate, malic acid, maltitol, maltitol solution, maltodextrin, maltol, maltose, mannitol, medium-chain triglycerides, meglumine, menthol, methylcellulose, methylparaben, mineral oil, mineral oil and lanolin alcohols, monoethanolamine, monosodium glutamate, monothioglycerol, myristic acid, neohesperidin dihydrochalcone, nitrogen, nitrous oxide, octyldodecanol, oleic acid, oleyl alcohol, olive oil, palmitic acid, paraffin, peanut oil, pectin, petrolatum, petrolatum and lanolin alcohols, phenol, phenoxyethanol, phenylethyl alcohol, phenylmercuric acetate, phenylmercuric borate, phenylmercuric nitrate, phosphoric acid, polacrilin potassium, poloxamer, polycarbophil, polydextrose, polyethylene glycol, polyethylene oxide, polymethacrylates, poly(methyl vinyl ether/maleic anhydride), polyoxyethylene alkyl ethers, polyoxyethylene castor oil derivatives, polyoxyethylene sorbitan fatty acid esters, polyoxyethylene stearates, polyvinyl acetate phthalate, polyvinyl alcohol, potassium alginate, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, 2-pyrrolidone, raffinose, saccharin, saccharin sodium, saponite, sesame oil, shellac, simethicone, sodium acetate, sodium alginate, sodium ascorbate, sodium benzoate, sodium bicarbonate, sodium borate, sodium chloride, sodium citrate dihydrate, sodium cyclamate, sodium hyaluronate, sodium hydroxide, sodium lactate, sodium lauryl sulfate, sodium metabisulfite, sodium phosphate, dibasic, sodium phosphate, monobasic, sodium propionate, sodium starch glycolate, sodium stearyl fumarate, sodium sulfite, sorbic acid, sorbitan esters (sorbitan fatty acid esters), sorbitol, soybean oil, starch, starch (e.g., pregelatinized, sterilizable maize), stearic acid, stearyl alcohol, sucralose, sucrose, sugar, compressible, sugar, confectioner's, sugar spheres, sulfobutylether b-cyclodextrin, sulfuric acid, sunflower oil, suppository bases, hard fat, talc, tartaric acid, tetrafluoroethane, thaumatin, thimerosal, thymol, titanium dioxide, tragacanth, trehalose, triacetin, tributyl citrate, triethanolamine, triethyl citrate, vanillin, vegetable oil, hydrogenated, water, wax, anionic emulsifying, wax (e.g., carnauba, cetyl esters, microcrystalline, nonionic emulsifying, white, yellow), xanthan gum, xylitol, zein, zinc acetate, zinc stearate, or any combination thereof.

In some aspects, a pharmaceutically acceptable excipient may comprise a carbohydrate, an alginate, a povidone, a carbomer, a flavor, a natural gum, a silicone, an alcohol, a butter, a wax, a fatty acid, a preservative, a pharmaceutically acceptable salt of any of these, or any combination thereof. In some aspects, a pharmaceutically acceptable excipient may comprise a carbohydrate. In some instances, the carbohydrate may comprise lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, a pharmaceutically acceptable salt of any of these, or any combination thereof. In some aspects, a pharmaceutically acceptable excipient may comprise lactose. In some instances, lactose may comprise milled lactose, sieved lactose, micronized lactose, spray dried lactose, at least substantially anhydrous lactose, monohy drate lactose, or a combination thereof. In some cases, a pharmaceutically acceptable excipient can comprise FDKP (fumaryl diketopiperazine) 1,2-distearoyl-sn-glycero-3-phosphocholine (DSPC), or both.

In some aspects, the active ingredient or pharmaceutically acceptable salt thereof may be contained at least in part within an excipient. In some aspects, the active ingredient or pharmaceutically acceptable salt thereof may be contained at least in part in an excipient. In some aspects, the active ingredient may be contained within a pore of an excipient. The “pore” of the excipient may refer to excipient particles that have been engineered to have open or closed pore structures. Porous excipient particles may be carriers of pharmaceutically active ingredients. Porous excipient particles may have a large surface area, stable structure, adjustable pore sizes, tunable dissolution, diffusion, or distribution, and well-defined surface properties. Porous excipient particles may facilitate sustained-release unit doses.

In some aspects, in addition to the active pharmaceutical ingredients or salts thereof, the compositions may further comprise inactive ingredients selected from the group consisting of microcrystalline cellulose, anhydrous dibasic calcium phosphate, croscarmellose sodium, magnesium stearate, hypromellose, titanium dioxide, lactose, triacetin, mannitol, xylitol, sorbitol, sugar alcohols, cellulose, cellulose esters, cellulose ethers, modified celluloses, starch, modified starches, polysaccharides, oligosaccharides, disaccharides, saccharides, gelatin, polyvinylpyrrolidone, polyethylene glycol, binders, flavorants, colorants, FD & C Blue #2 aluminum lake, magnesium stearate, anti-adherent agents, stearate salts, sweeteners, silica, lubricants, or any combination thereof.

### Inhalable Formulations

Advancements in engineering and process automation allow for enhanced methods of manufacturing to achieve consistent active ingredient particle size in the 1.0-10.0 micrometer size, which contributes to proper absorption in the lungs. A drug may be processed using spray drying technology to control the particle size and particle size distribution. Spray drying may be used to produce an active ingredient particle size in the 1.0-10.0-micrometer range. This particle size may be needed when a drug is administered by inhalation or by an intranasal route of administration for absorption into the lung alveolar. In some instances, the particle may be microencapsulated to enhance bioavailability. This route of administration may result in a rapid introduction of the drug into the blood stream and may require lower dosing when compared to oral intake of a capsule or tablet. For example, introducing encapsulated serotonin receptor agonists into the lungs via inhalation, may allow serotonin receptor agonists to reach the blood stream within 5 minutes.

In some aspects the compositions may comprise one or more of: an active ingredient or salts, excipients, and inactive ingredients. In some cases, a pharmaceutical composition may comprise particles. In some cases, particles may comprise an excipient (e.g., a pharmaceutically acceptable excipient), an active ingredient, an encapsulated active ingredient or any combination thereof. In some cases, the compositions may comprise a pharmaceutical composition. In some instances, a composition may comprise particles of a pharmaceutically acceptable excipient. In some instances, a composition may comprise particles of an active ingredient.

As used herein, “coating material” may refer to a material added via a pharmaceutical coating process by which an essentially dry, outer layer of coating material may be applied to the surface of a dosage form. Coating dosage forms may be used to improve stability (light protection, moisture and gas barrier), facilitate administration, or modify the drug release behavior from the dosage form. The coating materials may be used to enable the immediate release of the drug, delay the release of the drug (such as in enteric coatings), or sustain the release of the drug from the dosage form over extended periods of time. Coating materials may include film coating formulations, which usually contain a polymer, a plasticizer, a colorant, opacifier, a solvent, and a vehicle. In some cases, a coating material may refer to the coating material used in the coating of a particle of an active ingredient to create an encapsulated particle.

In some aspects, a composition may comprise a mixture of particles described herein. In some aspects, the particles may be mixed in a substantially homogenous mixture. In some instances, at least a portion of the particles of the pharmaceutically acceptable excipient may have a particle diameter ranging from about 50 micrometers to about 200 micrometers, as measured by a particle size analyzer using laser diffraction. In some cases, at least a portion of the active ingredient particles may have a particle diameter ranging from about 500 nanometers to about 15 micrometers, or about 1 micrometer to about 20 micrometers, as measured by a particle size analyzer using laser diffraction. In some instances, in a human clinical trial, the powdery pharmaceutical composition, when inhaled into the lungs, may provide in at least part of the humans in the clinical trial a time to peak plasma concentration (Tmax) of the active ingredient or the salt thereof ranging from about 1 minute to about one hour, or from about 1 minute to about ten minutes. In some aspects, the Tmax of the active ingredient or the salt thereof ranging from about 1 min to about 5 min, about 1 min to about 10 min, about 1 min to about 20 min, about 1 min to about 25 min, about 1 min to about 30 min, about 1 min to about 40 min, about 1 min to about 50 min, about 1 min to about 60 min, about 5 min to about 10 min, about 5 min to about 20 min, about 5 min to about 25 min, about 5 min to about 30 min, about 5 min to about 40 min, about 5 min to about 50 min, about 5 min to about 60 min, about 10 min to about 20 min, about 10 min to about 25 min, about 10 min to about 30 min, about 10 min to about 40 min, about 10 min to about 50 min, about 10 min to about 60 min, about 20 min to about 25 min, about 20 min to about 30 min, about 20 min to about 40 min, about 20 min to about 50 min, about 20 min to about 60 min, about 25 min to about 30 min, about 25 min to about 40 min, about 25 min to about 50 min, about 25 min to about 60 min, about 30 min to about 40 min, about 30 min to about 50 min, about 30 min to about 60 min, about 40 min to about 50 min, about 40 min to about 60 min, or about 50 min to about 60 min.

In some aspects, when inhaled into the lungs in a human clinical trial, the powdery pharmaceutical composition operates mechanistically such that in at least a portion of the humans in the clinical trial, a majority of the particles of the pharmaceutically acceptable excipient deposit onto the oropharynx.

In some aspects, the weigh to weight ratio of: a) the particles of the pharmaceutically acceptable excipient and b) the active ingredient particles ranges from about 1:1 to about 10000:1. In some aspects, the weight to weight ratio of: a) the particles of the pharmaceutically acceptable excipient and b) the active ingredient particles ranges from about 1:1 to about 20:1, about 1:1 to about 15:1, about 1:1 to about 10:1, about 1:1 to about 5:1, about 1:1 to about 2:1, about 2:1 to about 20:1, about 2:1 to about 15:1, about 2:1 to about 10:1, about 2:1 to about 5:1, about 5:1 to about 20:1, about 5:1 to about 15:1, about 5:1 to about 10:1, about 10:1 to about 15:1, about 10:1 to about 20:1, about 15:1 to about 20:1, about 18:1 to about 25:1, or about 25:1 to about 30:1. In some aspects, the weight to weight ratio of: a) the particles of the pharmaceutically acceptable excipient and b) the active ingredient particles may be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1 In some aspects, the weight to weight ratio of: a) the particles of the pharmaceutically acceptable excipient and b) the active ingredient particles ranges from about 1:1 to about 1:10, about 1:1 to about 1:8, about 1:1 to about 1:5, about 1:1 to about 1:2, about 1:2 to about 1:10, about 1:2 to about 1:8, about 1:2 to about 1:5, about 1:5 to about 1:10, about 1:5 to about 1:8, about 1:8 to about 1:10.

In some aspects, at least a portion of the particles of the pharmaceutical excipient and the active ingredient particles may not be covalently bound to each other.

### Orally Administered Microencapsulated Formulations

Disclosed herein are devices, systems and methods for producing, packaging, and delivering stable powdery compositions via oral administration. In some cases, a microencapsulated particle can contain multiple coatings and be orally administered. For example, a microencapsulated particle with one or more coatings (e.g., shells) can be added to a liquid and administered orally. In some embodiments, a microencapsulated particle can be configured (for example, with one or more layers of an enteric coating) to at least partially release an active ingredient in: the mouth, the esophagus, the small intestine, the duodenum, the jejunum, the ileum, the cecum, the colon, the ascending colon, the traverse colon, the descending colon, the sigmoid colon, the rectum, the anus, or any combination thereof. In some cases, microencapsulated particles can be added to a capsule, such as a capsule in capsule composition and be orally administered.

In some embodiments, the compositions can be spray dried. In some embodiments, the spray dried powder can be processed through a fluid bed to apply a polymer barrier or enteric coating. In some embodiments, the compositions can comprise one or more coatings. For example, a microencapsulated particle can have 1, 2, 3, 4, 5, or more than 5 coatings comprising the same or different coating material. Overall, the spray dried particles can individually have a particle diameter ranging from about 20 micrometers to about 200 micrometers or have a mean or median particle diameter of about 20 micrometers to about 200 micrometers, as measured by a particle analyzer using laser diffraction.

In some cases, a first coating may comprise a hydroxypropyl methylcellulose (HPMC), a hydroxypropyl methylcellulose acetate succinate (HPMCAS), a cyclodextrin, a maltodextrin, a povidone, a copovidone, or any combination thereof. In some cases, a first coating can comprise any wall or shell material.

In some cases, the first coating can be substantially encapsulated by one or more enteric coatings. An enteric coating comprises a barrier, such as a polymer barrier, that can be applied to a composition (for example a microencapsulated particle) to prevent dissolution or disintegration in the stomach. In some cases, this can enable the active ingredient to bypass the stomach to the small intestines before the active ingredient is released. In some cases, a wall material such as an additional coating on a previously microencapsulated particle can comprise an enteric coating. In some cases, the first coating of a microencapsulated particle can comprise an enteric coating. In some cases, an enteric coating can comprise methyl methacrylate (MMA). In some cases, an enteric coating can comprise a plant fiber, a shellac, a wax, s fatty acid, a plastic, or a combination thereof. In some cases, an enteric coating can comprise a methyl acrylate-methacrylic acid copolymer, a cellulose acetate phthalate (CAP), a cellulose acetate succinate, hydroxypropyl methyl cellulose phthalate, a HPMC-AS), a polyvinyl acetate phthalate (PVAP), a methyl methacrylate-methacrylic acid copolymer, a shellac, a cellulose acetate trimellitate, a sodium alginate, a zein, an enteric coating solution (an ethylcellulose, a medium chain triglycerides, an oleic acid, a sodium alginate, a stearic acid), or a combination thereof.

In some instances, a wall material coating can comprise an enteric coating, a time release coating, a pH dependent coating, a delayed release coating, an extended-release coating, or a combination thereof. The wall material can be biodegradable and biocompatible with the pharmaceutical ingredient. In some cases, the wall material can be biodegradable and biocompatible with a previously applied wall material.

In some cases, a microcapsule can be produced by dissolving, dispersing, or mixing the pharmaceutical ingredient in a solvent containing the shell material to produce a liquid suspension. In some cases, a multiple coated microcapsule can be produced by dissolving, dispersing, or mixing a previously microencapsulated pharmaceutical ingredient in a solvent containing a second shell material to produce a liquid suspension. For example, HPMCAS can be dissolved with ethanol and water and a pharmaceutical compound (e.g., the core) can be added the liquid suspension. In another example, an active ingredient encapsulated by HPMCAS can be dispersed with water and an enteric shell can be added to the liquid suspension. In some instances, the pharmaceutical compound may not dissolve in the liquid suspension. In some instances, the pharmaceutical compound may dissolve in the liquid suspension. In some instances, an encapsulated particle may not dissolve in the liquid suspension. In some instances, an encapsulated particle may dissolve in the liquid suspension. A liquid suspension can be dried with a spray drying technique described herein or by another method.

One or more layers of an enteric coating can be added to an active ingredient described herein by a microencapsulation process and/or fluidized system described herein to prevent it from dissolving until after it passes through the stomach. In some cases, two or more enteric coatings can be applied to an active ingredient. In some cases, a microencapsulated particle coating can release an active ingredient depending on the pH value within the gastrointestinal (GI) tract. The GI tract can have different pH values which can allow for pH dependent dosing in specific areas. For example, the pH of the stomach (acidic about 1.5-4.0 pH) is different from the pH of the small intestine (pH 4.0-7.0), and a pH microencapsulated particle coating can be used to dose areas of the GI tract with specific pH levels. In some embodiments, an enteric coating of a microencapsulated particle can be a polymer barrier that can be applied to the microencapsulated particle described herein to enable a controlled release. Bypassing the stomach can allow for more precise dosing and can enable the drug to achieve a higher bioavailability in the gastric tract. In some cases, these coatings or multiple layers of these coatings can be modified to deliver medicine from the mouth, all the way to the colon. In some cases, the technology can be applied to different microencapsulated layers of an active ingredient particle and utilize time-released, pH-controlled released, or a combination of both technologies to achieve the intended drug delivery. In some cases, one or more layers of a microcapsule shell can increase or decrease active ingredient release kinetics. In some cases, one or more layers of a microcapsule shell can increase or decrease bioavailability. In some cases, microencapsulation of an active ingredient as disclosed herein such as psilocin, can produce about: 5% to about 70%, 5% to about 10%, 5% to about 20%, 10% to about 30%, 15% to about 40%, 25% to about 40%, 10 % to about 60%, or 20% to about 50% more bioavailability of the active ingredient or the salt thereof as compared to the active ingredient or the salt thereof that is not encapsulated when ingested by a subject.

In terms of thickness of an individual coating of a microencapsulated particle, the thickness can be more than, less than, or equal to about: 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases, the wall thickness can of an individual coating of a microencapsulated particle can range from about: 500 nm to about 1 μm, 1 μm to about 10 μm, 1 μm to about 5 μm, 2 μm to about 7 μm, 3 μm to about 8 μm, 5 μm to about 10 μm, 5 μm to about 15 μm, or 1 μm to about 30 μm.

In some instances, the wall thickness of a microencapsulated particle can increase by increasing the ratio of the wall material to a previously microencapsulated particle prior to spray drying. In some cases, the ratio of a wall material to a previously microencapsulated particle (weight/weight) can be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 60:1, 70:1, 80:1, 90:1, or 100:1. In some cases, the ratio of a previously microencapsulated particle to a wall material (weight/weight) can be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 60:1, 70:1, 80:1, 90:1, or 100:1.

In certain instances, the formulations may be such that not all microencapsulated particles have the same number of coatings. This may be advantageous wherein delivery of an active ingredient may take place at multiple points along the digestive tract. In some cases, in a plurality of microencapsulated particles about: 1% to about 50%, 1% to about 20%, 1% to about 10%, 5% to about 25%, 10% to about 40%, 10% to about 60%, 20% to about 70%, 20% to about 50%, 30% to about 80%, 40% to about 90%, 50% to about 75%, 60% to about 80%, 70% to about 90%, 75% to about 95%, 80% to about 90%, 80% to about 99%, 85% to about 100%, or 90 % to about 100% of the microencapsulated particles can comprise two or more coatings.

In some cases, the mixed sizes or mixed number of coatings can change the release time of the drug. For example, encapsulated particles that comprise an additional enteric coating with small sizes (e.g., about 20 μm to about 40 μm) can be readily absorbed from the intestines into the blood stream while larger enteric coated, encapsulated particles larger than about 60 μm can take longer to be absorbed into the blood stream. In some cases, particles with diameters of about 20 μm to about 40 μm can absorb faster than particles with diameters of about 50 μm to about 200 μm. In some embodiments, the particles with sizes of about 50 μm to about 200 μm can be mixed with particles with sizes of about 20 μm to about 40 μm. In some embodiments, the weight to weight ratio of the particles with diameters of about 70 μm to about 100 μm to the particles with sizes of about 30 μm to about 60 μm can range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some embodiments, the weight to weight ratio of the particles with diameters of about 70 μm to about 100 μm to the particles with sizes of about 30 μm to about 60 μm can range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some embodiments, the particles with larger sizes (about 70 μm to about 200 μm) can be mixed with particles with smaller sizes (about 20 μm to about 40 μm). In some embodiments, the weight to weight ratio of the particles with larger sizes (about 70 μm to about 200 μm) to the particles with smaller sizes (about 20 μm to about 40 μm) can be ranging from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10.

### Method of Making the Powdery Pharmaceutical Composition

In some cases, methods of making a pharmaceutical composition may comprise creating particles by the methods described herein. In some cases, particles may comprise an excipient (e.g., a pharmaceutically acceptable excipient), an active ingredient, or both. In some aspects, a method of making a powdery pharmaceutical composition, may comprise mixing, in a mixer, or contacting particles of a pharmaceutically acceptable excipient; and particles comprising an active ingredient or a pharmaceutically acceptable salt thereof. In some cases, the particles comprising an active ingredient may be microencapsulated. For example, particles of a serotonin receptor agonist may be microencapsulated with a HPMC or HPMCAS coating. In some instances, at least a portion of the particles of the pharmaceutically acceptable excipient may have a particle diameter ranging from about 50 micrometers to about 200 micrometers, as measured by a particle size analyzer using laser diffraction. In some cases, particles comprising the active ingredient may have a particle diameter ranging from about 500 nanometers to about 15 micrometers, or 1 micrometer to about 20 micrometers, as measured by a particle size analyzer using laser diffraction.

In some aspects, a method of making the powdery pharmaceutical composition may comprise spray drying particles. In some cases, the particles can comprise an active ingredient or a pharmaceutically acceptable salt thereof. In some aspects, the spray drying process may comprise: atomizing liquid droplets comprising the active ingredient or the pharmaceutically acceptable salt thereof, drying the droplets from particles, recovering the particles, or any combination thereof.

In some aspects, a spray drying manufacturing system may comprise a closed spray dryer container which receives the solution comprising a drug dissolved or mixed in a suitable solvent (aqueous or solvent based). In some cases, a solvent may comprise alcohol, ethanol, dimethylformamide (DMF), dimethyl sulfoxide (DMSO), a polar organic solvent, an organic solvent, or any combination thereof. In some aspects, the solution then enters the particle formation chamber which may be connected to an atomizer located at the top of the chamber. In some aspects, the atomizer may be a two component or rotary nozzle type that distributes the solution into fine droplets controlled by the atomizer pressure. In some aspects, this atomization gas may be an inert gas. As used herein, “inert gas” may refer to a non-reactive gas, or a gas that does not undergo chemical reactions under a set of given conditions. Inert gases may be generally used to avoid unwanted chemical reactions degrading a sample, or to prevent bacterial growth. These undesirable chemical reactions may often be oxidation and hydrolysis reactions with the oxygen and moisture in air. The term “inert gas” may be context-dependent because several of the noble gases, which have been historically referred to as the inert gases, may be made to react under certain conditions. In some aspects, inert gas may be air, nitrogen, carbon dioxide or any combination thereof. In some aspects, the atomized droplets go through a hot gas drying chamber to produce uniform fine particles that maintain a tight particle size distribution following liquid evaporation. In some cases, the solid particle forms and falls to the bottom of the drying chamber. In some instances, the balance between temperature, flow rate, and droplet size may controls the drying process. In some aspects, the powder may be recovered from the exhaust gas using a cyclone or a bag filter. In some aspects, particle size may be validated by a Malvern particle analyzer prior to blending with an excipient carrier. In some aspects, the active powder (e.g., the powdery pharmaceutical composition) may be blended with an excipient carrier (lactose) product in a Patterson Kelly (PK Blender) and the blended powder may be fed to a hopper. In some aspects, from the hopper, the dry powder may be placed into a Size 3 hypromellose capsule, by a Bosch Encapsulator machine. In some cases, the dry powder may be placed into any capsule of any size. For example, the dry powder may be placed into a size 000, 00, 0, 1, 2, 3, or a 4 size capsule.

In some aspects, a method of spray drying a liquid is disclosed herein. In some cases, a liquid may comprise i) a serotonin agonist, or a pharmaceutically acceptable salt thereof; ii) a coating material, wherein the coating material may comprise a hydroxypropyl methylcellulose (HPMC), a hydroxypropyl methylcellulose acetate succinate (HPMCAS), a cyclodextrin, a maltodextrin, a povidone, a copovidone or any combination thereof; and iii) a solvent. In some cases, the particles of the serotonin agonist or the pharmaceutically acceptable salt thereof may be dispersed in the liquid. In some cases, the particles of the serotonin agonist or the pharmaceutically acceptable salt thereof dispersed in the liquid may have a particle diameter ranging from about 1 micrometer to about 5 micrometers. In some instances, the spray drying may comprise i) atomizing liquid droplets comprising the serotonin agonist or the pharmaceutically acceptable salt thereof, the coating material, and the solvent, ii) drying the droplets to form substantially encapsulated particles wherein the substantially encapsulated particles may comprise the serotonin agonist or the pharmaceutically acceptable salt thereof substantially encapsulated by the coating material and iii) recovering the substantially encapsulated particles.

In some aspects, a method of making a powdery pharmaceutical composition, may comprise blending and/or contacting: i) particles of a pharmaceutically acceptable excipient; and ii) a plurality of spray dried particles. In some cases, each particle of the plurality of spray dried particles may comprise a serotonin receptor agonist such as psilocybin or a pharmaceutically acceptable salt thereof. In some cases, the serotonin receptor agonist can be substantially encapsulated in a coating material. In some cases, a portion of the plurality of spray dried particles comprising the serotonin receptor agonist or the pharmaceutically acceptable salt thereof substantially encapsulated in the coating material may have a particle diameter ranging from about 1 micrometer to about 10 micrometers, or from about 1 micrometer to about 5 micrometers, as measured by a particle analyzer using laser diffraction. In some instances, the coating material may comprise a hydroxypropyl methylcellulose (HPMC), a hydroxypropyl methylcellulose acetate succinate (HPMCAS), a cyclodextrin, a maltodextrin, a povidone, a copovidone or any combination thereof.

In some aspects, a powdery pharmaceutical composition may be produced by a process comprising: a) contacting the particles comprising a serotonin receptor agonist (e.g., psilocybin or a pharmaceutically acceptable salt thereof), a coating material, and a solvent and b) spray drying the mixed particles comprising the serotonin receptor agonist, the coating material, and the solvent. In some cases, the spray dried particles may be mixed or blended with a pharmaceutically acceptable excipient to make a powdery pharmaceutical composition.

The moisture level of the powder after spray drying may be below about 10%. In some aspects, the moisture level may be below about 15%, about 14%, about 13%, about 12%, about 11%, about 10%, about 9%, about 8%, about 7%, about 6%, about 5%, about 4%, about 3%, about 2%, or about 1%.

In some embodiments, the method of making a composition can comprise formulating the particles described herein into a capsule-in-capsule composition (e.g. a pharmaceutical composition). In some cases, particles can comprise an excipient, an active ingredient, or both. In some cases, particles can comprise a carrier, an active ingredient, or both. In some cases, particles can comprise a diluent, an active ingredient, or both. In some embodiments, a capsule-in-capsule formulation can be in unit dose form. In some cases, a formulation can comprise particles comprising N-lactoyl-phenylalanine or a pharmaceutically acceptable salt thereof. In some instances, the particles can be at least partially encapsulated by a coating material. In some instances, the particles at least partially encapsulated by the coating material can be spray dried. In some cases, the particles can be at least partially surrounded by a first capsule, a second capsule, or both. In some instances, the first capsule can be surrounded by a second capsule to create a capsule-in-capsule, capsule. In some cases, a capsule can comprise a capsule coating. In some cases, a capsule coating can at least partially control capsule ingredient release.

In some embodiments, a final product can be a capsule-in-capsule. In some cases, the final product can be a capsule (e.g. a second capsule) that surrounds an active ingredient (e.g., a serotonin receptor agonist) and separately an inner capsule (e.g. the first capsule), which can contain its own active ingredient. In some instances, a capsule can contain more than one active ingredient. In some instances, a capsule can contain more than one inner capsule. For example, a capsule can comprise 1, 2, 3, 4, 5, 6, 7, 8, 9, 10 or more inner capsules. In some cases, an inner capsule can comprise a capsule. For example, an outer capsule can surround a first inner capsule and the first inner capsule can surround a second inner capsule. In some cases, an active ingredient can comprise extracted psilocybin or any extracted ingredient disclosed herein. In some cases, the active ingredient can be microencapsulated and spray dried. In some cases, the active ingredient can be spray dried but not microencapsulated.

The process described herein can include the following manufacturing stages. The active ingredient of the first capsule and the second capsule can be microencapsulated and spray dried using the methods described herein. In some embodiments, the active ingredients can be independently blended with an excipient. In some cases, the active ingredients may not be blended with an excipient. In some cases, the active ingredient of the first capsule can then be added to the first capsule and the first capsule can be banded using the methods described herein. In some cases, after the first capsule is banded, a capsule coating (e.g. an enteric, pH dependent, time release, or combination release) can be applied to the first capsule. In some cases, the active ingredient of the second capsule can then be added to the second capsule and the first capsule can be placed into the second capsule. The second capsule can be banded, and a capsule coating can be applied to the second capsule.

In some embodiments, a capsule can further comprise a capsule coating. In some cases, a capsule coating can be added to a capsule to further improve stability (light protection, moisture and gas barrier), facilitate administration, or modify the composition release behavior from the dosage form. A capsule coating may be used to enable the immediate release of the composition, delay the release of the composition (such as in enteric coatings), or sustain the release of the composition over extended periods of time. In some instances, a capsule coating can comprise a film coating, a gelatin coating, or both. In some instances, a capsule coating can comprise an enteric coating, a time release coating, a pH dependent coating, a delayed release coating, an extended release coating, or a combination thereof. For example, an enteric coating can be added to a capsule to prevent it from dissolving until after it passes through the stomach. In some cases, the composition can release depending on the pH value within the gastrointestinal (GI) tract. The GI tract can have different pH values which can allow for pH dependent dosing in specific areas. For example, the pH of the stomach (acidic about 1.5-4.0 pH) is different from the pH of the small intestine (pH 4.0-7.0), and a pH coating can be used to dose areas of the GI tract with specific pH levels. In some embodiments, an enteric coating of a capsule can be a polymer barrier that can be applied to the capsules described herein to enable a controlled release. Bypassing the stomach can allow for more precise dosing and can enable the drug to achieve a higher bioavailability in the gastric tract. In some cases, these coatings can be modified to deliver medicine from the mouth, all the way to the colon. In some cases, the technology can be applied to the outer (e.g. the second capsule) and the inner (e.g. the first capsule) capsule in the capsule-in-capsule technology and utilize time-released, pH-controlled released, or a combination of both technologies to achieve the intended drug delivery. In some instances, an enteric coating can be applied to multiple capsules, for example to an inner capsule and to an outer capsule and to provide delayed release of both capsules. In some cases, a capsule coating can provide a color, mask a bitter taste, or both. In some cases, a capsule coating can comprise polymers, plasticizers, pigments, opacifiers, glidants, binders, anti-tacking agents, anti-foaming mechanisms, surfactants, fillers, and extenders.

In some embodiments, an enteric coating can comprise a polymer. In some cases, an enteric coating can comprise a methyl acrylate-methacrylic acid copolymer, a cellulose acetate phthalate, methyl methacrylate (MMA), a cellulose acetate succinate, a hydroxypropyl methyl cellulose phthalate, a hydroxypropyl methyl cellulose acetate succinate (hypromellose acetate succinate), a polyvinyl acetate phthalate, a methyl methacrylate-methacrylic acid copolymers, a shellac, a cellulose acetate trimellitate, a sodium alginate, a zein, an ethylcellulose, a medium chain triglycerides, an oleic acid, a stearic acid or any combination thereof.

In some embodiments, a capsule can be configured (for example with a capsule coating) to at least partially release an active ingredient in: the mouth, the esophagus, the small intestine, the duodenum, the jejunum, the ileum, the cecum, the colon, the ascending colon, the traverse colon, the descending colon, the sigmoid colon, the rectum, the anus, or any combination thereof.

In some cases, a composition can comprise a mixture of particles described herein. In some instances, at least a portion of an excipient and at least a portion of the particles comprising an active ingredient, can comprise a mixture or a formulation.

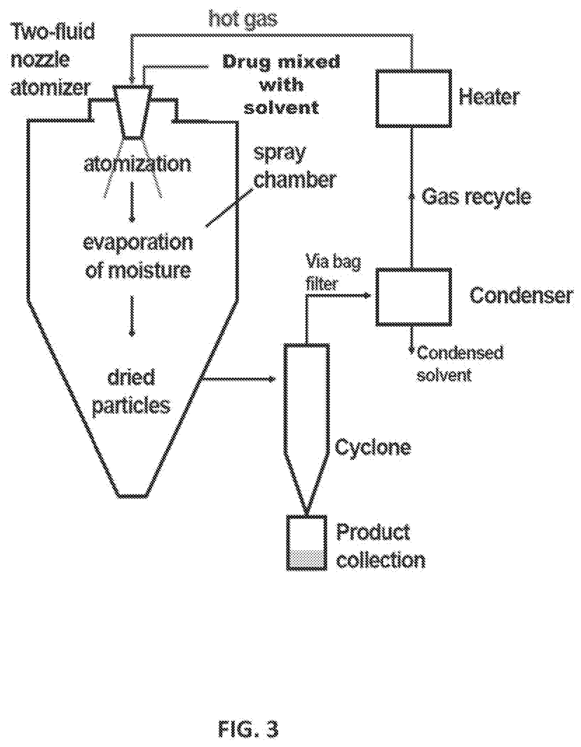

Referring to FIG. 3, FIG. 3 shows a spray drying manufacturing system comprising a closed spray dryer container which receives the solution comprising a drug dissolved or mixed in a suitable solvent (aqueous or solvent based). The solution then enters the particle formation chamber which is connected to an atomizer located at the top of the chamber. The atomizer is a two component or rotary nozzle type that distributes the solution into fine droplets controlled by the atomizer pressure. This atomization gas is an inert gas, either air or nitrogen. The atomized droplets go through a hot gas drying chamber to produce uniform fine particles that maintain a tight particle size distribution following liquid evaporation. Solid particles form and fall to the bottom of the drying chamber. The balance between temperature, flow rate, and droplet size, controls the drying process. The powder is recovered from the exhaust gas using a cyclone or a bag filter. Particle size is validated by a Malvern particle analyzer prior to blending with an excipient carrier. The active powder is blended with an excipient carrier (lactose) product in a Patterson Kelly (PK Blender) and the blended powder is fed to a hopper. From the hopper, the dry powder is placed into a Size 3 Hypromellose capsule, by a Bosch Encapsulator machine.

### Microencapsulation

In some aspects, encapsulation may comprise microencapsulation. Microencapsulation may be a process in which a microcapsule may be created as a small sphere or multi-sphere in one core with a matrix wall around it. The pharmaceutical ingredient inside the microcapsule may be called a fill. In some cases, a fill may be a liquid, an oil, a solid or any combination thereof. The wall around the fill (“or core”) may be referred to as a shell, a coating, or a membrane. Microcapsules may have a diameter as small as 1.0 micron in size to about 10.0 micron in size or about 1.0 micron to about 5.0 microns in size. In some cases, the small size may provide a pharmaceutical ingredient a large surface area to be available for absorption, release, transfer, or any combination thereof. In some cases, microencapsulation may at least partially prevent inhalation of an active ingredient comprising the form of an unencapsulated crystal. For example, microencapsulation may at least partially prevent inhalation of unencapsulated crystals comprising a serotonin receptor agonist. In some instances, unencapsulated crystals such as serotonin receptor agonist crystals may cause irritation of the respiratory tract of a subject during inhalation. The irritation may be caused by crystal geometry and structure. For example, a crystal may have sharp angles and edges that may cause irritation, damage or both of the respiratory tract during inhalation. In some instances, crystal geometry and structure may be controlled by the spray drying process. Microencapsulation may generate crystals with amorphous structure. In some instances, an amorphous crystal may lack sharp edges and angles. In some cases, an amorphous crystal may have a rounded edge. In some instances, an amorphous crystal may have increased bioavailability.

In some instances, an agonist described herein comprised in an oil may be microencapsulated with compatible diluents to protect the oil from oxidation and provide a longer shelf life than the unprotected pharmaceutical composition. Similarly, a serotonin receptor agonist may be encapsulated to provide a longer shelf life. The diluents may be aqueous, or solvent based and use animal or plant materials. In some cases, the diluent may comprise alcohols: e.g., ethanol, butanol, 2-ethylhexanol, isobutanol, isopropanol, methanol, propanol, propylene glycol; ketones: e.g., acetone, methyl ethyl ketone, methyl isobutyl ketone, methyl isopropyl ketone, mesityl oxide, trichloroethylene; halogenated solvents: e.g., ethylene bromide, chloroform, ethylene chloride, dichloromethane, tetrachloroethylene, carbon tetrachloride; amides: e.g., dimethylformamide; ethers: e.g., 1,4-dioxane, butyl ether, ethyl ether, di-isopropyl ether, tetrahydrofuran, tert-butyl methyl ether; sulfur containing solvent: e.g., dimethyl sulfoxide; amines: e.g.,, pyridine; nitriles: e.g., acetonitrile; esters: e.g., ethyl acetate; aliphatic hydrocarbons: e.g., cyclohexane hexane; aromatic hydrocarbons: e.g., toluene xylene; water or any combinations thereof. In some cases, the diluent may comprise benzene, carbon tetrachloride, 1,2-dichloroethane, 1,1-dichloroethene, 1,1,1-trichloroethane, acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethylene glycol, formamide, hexane, methanol, 2-methoxyethanol, methylbutylketone, methylcyclohexane, n-methylpyrrolidone, nitromethane, pyridine, sulfolane, tetralin, toluene, 1,1,2-trichloroethylene, xylene or any combinations thereof.

The core active ingredient may be microencapsulated with an amphipathic molecule that has both a polar end (‘hydrophilic”) and non-polar end (“hydrophobic”). In some cases, a hydrophilic end of an amphipathic molecule may interact with core material. In some cases, a hydrophobic end of an amphipathic molecule may interact with core material. This hydrophilic and hydrophobic structure may enable the molecule to microencapsulate an active ingredient and form a microsphere. In some instances, the microencapsulated particle may have a hydrophilic exterior and a hydrophobic interior. In some instances, the microencapsulated particle may have a hydrophobic exterior and a hydrophilic interior. The microencapsulation process may coat the active ingredient, which is the core, by the amphipathic encapsulating agent, which is the wall material, so that the active ingredient is at least partially surrounded by a wall of the amphipathic material. For example, hydroxypropyl methylcellulose acetate succinate (HPMCAS) may be an amphipathic molecule used to coat a serotonin receptor agonist. The microencapsulation blend may be a spray dried dispersion, that may be fed into a spray dry system to create a hard-outer coating on the microcapsules.

The wall material may form a film that is cohesive with the core active ingredient. A wide variety of coating materials are available for encapsulation, e.g., traditional coating materials like inert polymers and pH sensitive ones as carboxylate and amino derivatives, which swell or dissolve according to the degree of cross-linking; some innovative coating polymers have also been developed for applications particularly among the bioadhesives and mucoadhesives. In some cases, the coating material may be hydrophilic polymers, hydrophobic polymers or a combination of both. In some cases, a microcapsule shell may comprise an amphipathic molecule. In some cases, the coating material may be a gelatin, a polyvinyl alcohol, an ethyl cellulose, a cellulose acetate phthalate or a styrene maleic anhydride. In some instances, the coating material may not react with the pharmaceutical ingredient. In some cases, a microcapsule shell may comprise a hydroxypropyl methylcellulose (“HPMC”), a hydroxypropyl methylcellulose acetate succinate (“HPMCAS”), a cyclodextrin, a maltodextrin, a povidone, a copovidone and others. In some instances, a microcapsule shell may comprise HPMCAS-LG, HPMCAS-MG, HPMCAS-HG or HPMC-P or a combination thereof. In some instances, a microcapsule shell may comprise a different grade of HPMC or HPMCAS. For example, a microcapsule shell may comprise an E5, an E50, or a K4M grade of HPMC. In another example, a microcapsule shell may comprise a L, a M, or an H grade of HPMCAS. In some cases, a microcapsule shell may comprise a HPMCAS. In some cases, a microcapsule shell may comprise a gelatin, a cornstarch, a polyvinylpyrrolidone (PVP), an oligosaccharide, a starch, a cellulose, a glycogen, a long chain sugar or any combination thereof. In some cases, a microcapsule shell may comprise FDKP (fumaryl diketopiperazine). In some cases, a microcapsule shell can comprise 1,2-distearoyl-sn-glycero-3-phosphocholine (DSPC). In some cases, a microcapsule shell can contain a weight to weight ratio of the DSPC, and the FDKP of about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 55:1, 60:1, 65:1, 70:1, 75:1, 80:1, 85:1, 90:1, 95:1, or 100:1 (DSPC to FDKP). In some cases, a microcapsule shell can contain a weight to weight ratio of the FDKP, and the DSPC of about: 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:55, 1:60, 1:65, 1:70, 1:75, 1:80, 1:85, 1:90, 1:95, or 1:100 (FDKP to DSPC).

In some cases, a microcapsule shell may comprise a fatty acid, a liposome, an amino acid, a natural oil and a sugar, a trehalose, a dextran, a natural oil, a synthetic oil or a combination thereof. In some cases, a sugar can comprise a dextrose, a fructose, a galactose, a glucose, a lactose, a maltose, a sucrose, a salt of any of these, or any combination thereof. In some instances, an amino acid may comprise a glutamic acid, an aspartic acid, a lysine, a tryptophan, a tyrosine, a methionine or a combination thereof. In some cases, a fatty acid may comprise a polyunsaturated fatty acid, an essential fatty acid, a conjugated fatty acid, a short chain fatty acid, a medium chain fatty acid, a long chain fatty acid, a very long chain fatty acid, a saturated fatty acid, an unsaturated fatty acid, a monounsaturated fat, or any combination thereof. In some cases, a fatty acid may comprise an omega-3 fatty acid, an omega-5 fatty acid, an omega-6 fatty acid, an omega-7 fatty acid, an omega-9 fatty acid, an omega-10 fatty acid, an omega-11 fatty acid, an omega-12 fatty acid, or a combination thereof. In some cases, a natural oil may comprise soy bean oil, a vegetable oil, a food oil, evening primrose oil, borage oil, blackcurrant seed oil, flax or linseed oil, rapeseed or canola oil, corn oil, almond oil, avocado oil, brazil nut oil, canola oil, cashew oil, chia seed oil, cocoa butter oil, coconut oil, corn oil, cottonseed oil, flaxseed/linseed oil, grape seed oil, hemp seed oil, vigna mungo oil, mustard oil, olive oil, palm oil, peanut oil, pecan oil, perilla oil, rice bran oil, safflower oil, sesame oil, soybean oil, walnut oil, sunflower oil, cottonseed oil, palm oil, or a combination thereof. In some cases, a microcapsule shell may increase or decrease active ingredient release kinetics. In some cases, a microcapsule shell may increase or decrease bioavailability. In some cases, microencapsulation of a serotonin receptor agonist, may produce about: 5% to about 70%, 5% to about 10%, 5% to about 20%, 10% to about 30%, 15% to about 40%, 25% to about 40%, 10 % to about 60%, or about 20% to about 50% more bioavailability of the serotonin receptor agonist thereof as compared to a serotonin receptor agonist that is not encapsulated when inhaled by a subject. The wall material may be biodegradable and biocompatible with the pharmaceutical ingredient. In some cases, a microcapsule may be produced by dissolving or mixing the pharmaceutical ingredient in a solvent containing the shell material to produce a liquid suspension. For example, HPMCAS may be dissolved with ethanol and water and a pharmaceutical compound may be added the liquid suspension. In some instances, the pharmaceutical compound may not dissolve in the liquid suspension. In some instances, the pharmaceutical compound may dissolve in the liquid suspension. The liquid suspension may be dried with a spray drying technique described herein or by another method.

In some cases, the average wall thickness may of a microencapsulated particle can be more than, less than, or equal to about: 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases, the wall thickness may of a microencapsulated particle may range from about: 1 μm to about 10 μm, 1 μm to about 5 μm, 2 μm to about 7 μm, 3 μm to about 8 μm, 5 μm to about 10 μm, 5 μm to about 15 μm, or 1 μm to about 30 μm. In some instances, the wall thickness of a microencapsulated particle may increase by increasing the ratio of the wall material to the core material prior to spray drying. In some cases, the ratio of wall material to a core material (weight/weight) may be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 60:1, 70:1, 80:1, 90:1, or 100:1. In some cases, the ratio of the wall material to core material (weight/weight) may be about 10:1.

In some aspects, in a plurality of microencapsulated particles about: 1%, 5%, 10%, 15%, 20%, 25%, 30%, 35%, 40%, 45%, 50%, 55%, 60%, 65%, 70%, 75%. 80%, 90%, 95%, 99% or 100% of the microencapsulated particles may comprise a core substantially encapsulated by a wall material. In some cases, in a plurality of microencapsulated particles about: 1% to about 50%, 1% to about 20%, 1% to about 10%, 5% to about 25%, 10% to about 40%, 10% to about 60%, 20% to about 70%, 20% to about 50%, 30% to about 80%, 40% to about 90%, 50% to about 75%, 60% to about 80%, 70% to about 90%, 75% to about 95%, 80% to about 90%, 80% to about 99%, 85% to about 100%, or 90 % to about 100% of the microencapsulated particles may comprise a core substantially encapsulated by a wall material. In some cases, in a plurality of microencapsulated particles not all of the core material may be encapsulated by the wall material.

In some aspects, microencapsulated particles have a mean, a median, or a mode particle diameter of less than about: 500 nm, 1 μm, 2 μm, 3 μm, 4 μm, 5 μm, 6 μm, 7 μm, 8 μm, 9 μm, 10 μm, 11 μm, 12 μm, 13 μm, 14 μm, or 15 μm. In some aspects, microencapsulated particles have a mean, a median, or a mode particle diameter of more than about: 500 nm, 1 μm, 2 μm, 3 μm, 4 μm, 5 μm, 6 μm, 7 μm, 8 μm, 9 μm, 10 μm, 11 μm, 12 μm, 13 μm, 14 μm, or 15 μm. In some aspects, microencapsulated particles have a mean, a median, or a mode particle diameter ranging from about: 500 nm to about 5 μm, 1 μm to about 10 μm, 1 μm to about 5 μm, 2 μm to about 7 μm, 3 μm to about 8 μm, 5 μm to about 10 μm, or 5 μm to about 15 μm.

In some embodiments, microencapsulated particles have a mean, a median, or a mode particle diameter of less than about: 500 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 μm, 147 μm, 148 μm, 149 μm, 150 μm, 151 μm, 152 μm, 153 μm, 154 μm, 155 μm, 156 μm, 157 pm, 158 μm, 159 μm, 160 μm, 161 μm, 162 μm, 163 μm, 164 μm, 165 μm, 166 μm, 167 μm, 168 μm, 169 μm, 170 μm, 171 μm, 172 μm, 173 μm, 174 μm, 175 μm, 176 μm, 177 μm, 178 μm, 179 μm, 180 μm, 181 μm, 182 μm, 183 μm, 184 μm, 185 μm, 186 μm, 187 μm, 188 μm, 189 μm, 190 μm, 191 μm, 192 μm, 193 μm, 194 μm, 195 μm, 196 μm, 197 μm, 198 μm, 199 μm, or 200 μm,. In some embodiments, microencapsulated particles have a mean, a median, or a mode particle diameter of more than about: 500 nm, 1 μm, 2 μm, 3 μm, 4 μm, 5 μm, 6 μm, 7 μm, 8 μm, 9 μm, 10 μm, 11 μm, 12 μm, 13 μm, 14 pm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 pm, 147 μm, 148 μm, 149 μm, 150 μm, 151 μm, 152 μm, 153 μm, 154 μm, 155 μm, 156 μm, 157 μm, 158 μm, 159 μm, 160 μm, 161 μm, 162 μm, 163 μm, 164 μm, 165 μm, 166 μm, 167 μm, 168 μm, 169 μm, 170 μm, 171 μm, 172 μm, 173 μm, 174 μm, 175 μm, 176 μm, 177 μm, 178 μm, 179 μm, 180 μm, 181 μm, 182 μm, 183 μm, 184 μm, 185 μm, 186 μm, 187 μm, 188 μm, 189 μm, 190 μm, 191 μm, 192 μm, 193 μm, 194 μm, 195 μm, 196 μm, 197 μm, 198 μm, 199 μm, or 200 μm. In some embodiments, microencapsulated particles have a mean, a median, or a mode particle diameter ranging from about: 500 nm to about 5 μm, 1 μm to about 10 μm, 1 μm to about 5 μm, 2 μm to about 7 μm, 3 μm to about 8 μm, 5 μm to about 10 μm, 5 μm to about 15 μm, 10 μm to about 50 μm, 20 μm to about 100 μm, 30 μm to about 70, 50 μm to about 150, 70 μm to about 100 μm, 70 μm to about 140, 100 μm to about 180, or 120 μm to about 200 μm. In some cases, microencapsulated particles in a capsule in capsule formulation can be larger than microencapsulated particles for use in a dry powdered inhalable formulation.

The core material may be the material over which a coating is applied. Core material may be in form of solids or droplets of liquids and dispersions. In some cases, core material may comprise a serotonin receptor agonist. In some cases, core material may comprise another serotonin receptor agonist or a salt thereof. The composition of core material may vary and thus furnish definite flexibility and allow effectual design and development of the desired microcapsule properties. A substance may be microencapsulated for a number of reasons. Examples may include protection of reactive material from their environment, safe and convenient handling of the materials which may be otherwise toxic or noxious, taste masking, means for controlled or modified release properties means of handling liquids as solids, preparation of free flow powders and in modification of physical properties of the drug. For example, encapsulation may improve solubility and dissolution and therefore increase bioavailability of an active ingredient such as a serotonin receptor agonist. Microencapsulation may be used to increase the stability, improve the handling properties of compounds, facilitate higher bioavailability when reconstituted or administered, or any combination thereof. For example, a microencapsulated serotonin receptor agonist may not require refrigeration and may not lose efficacy when exposed to a high temperature for a period of time. In some cases, the shelf life of an encapsulated serotonin receptor agonist may be extended from about: 4, 5, 6, 7, 8, 9, 10, 11, 12, 13, 14, 15, 16, 17, 18, 19, 20, 21, 22, 23, 24 or more months as compared to a liquid formation comprising a serotonin receptor agonist. In some instances, an inhalable serotonin receptor agonist may not require a needle for administration which may eliminate the potential for a needle breaking during an emergency.

In some instances, the core diameter of a microencapsulated particle may be more than, less than, or equal to about: 100 nm (nanometer), 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases, the core diameter of a microencapsulated particle may range from about: 100 nm to about 250 nm, 100 nm to about 500 nm, 100 nm to about 1 μm, 500 nm to about 1 μm, 1 μm to about 10 μm, 1 μm to about 5 μm, 2 μm to about 7 μm, 3 μm to about 8 μm, 5 μm to about 10 μm, 5 μm to about 15 μm, or 1 μm to about 30 μm. In some instances, the core may comprise about: 1%, 5%, 10%, 15%, 20%, 25%, 30%, 35%, 40%, 45%, 50%, 55%, 60%, 65%, 70%, 75%. 80%, 90%, 95% or 99% of the total microcapsule content (e.g., total weight of the core and wall material). In some instances, the core may comprise about: 1% to about 50%, 1% to about 20%, 1% to about 10%, 5% to about 25%, 10% to about 40%, 10% to about 60%, 20% to about 70%, 20% to about 50%, 30% to about 80%, 40% to about 90%, 50% to about 75%, or 1% to about 99% of the total microcapsule content.

In some aspects, a method of microencapsulation may comprise at least partially dissolving the coating material (e.g., HPMC or HPMCAS) in a solvent such as a mix of ethanol and water. In some cases, particles of a serotonin receptor agonist may be added to the solution of the coating material and the solvent to create a suspension of the particles of a serotonin receptor agonist and the coating material dissolved in the solvent. In some instances, the serotonin receptor agonist may not dissolve in the suspension and may remain in suspension. In some instances, the serotonin receptor agonist may dissolve in the suspension. The suspension may be mixed to an at least partially uniform mixture and spray dried. The coating may at least partially encapsulate the psylocibin or salt thereof. In some cases, the serotonin receptor agonist may be amorphous. In some cases, the encapsulation of the serotonin receptor agonist may be a spherical, round, oval, or any shape structure.

### Mixtures of Active Agent Particles and Excipient Particles

Also disclosed herein are methods of formulating the powdery pharmaceutical compositions. In some cases, the formulating can comprising mixing particles of an excipient and an active agent. In some cases, the formulating can comprise making particles of a specific size. In some aspects, the method of making the powdery pharmaceutical composition may comprise mixing the particles described herein with an excipient. In some instances, at least a portion of the particles of a pharmaceutically acceptable excipient may have a particle diameter ranging from about 50 micrometers to about 200 micrometers, as measured by a particle size analyzer using laser diffraction. In some cases, at least a portion of the particles comprising an encapsulated serotonin receptor agonist may have a particle diameter ranging from about 500 nanometers to about 15 micrometers, or about 1 micrometer to about 20 micrometers, as measured by a particle size analyzer using laser diffraction.

In some aspects, at least a portion of the particles of a pharmaceutically acceptable excipient may have a particle diameter ranging from about: 30 μm (micrometers) to about 60 μm, 50 μm, to about 200 μm, 60 μm to about 80 μm, 70 μm to about 100 μm. 90 μm to about 130 μm, 110 μm to about 150 μm, 130 μm to about 180 μm, 150 μm to about 200 μm, 190 μm to about 250 μm, or 200 μm to about 400 μm. In some cases, at least a portion of particles of a pharmaceutically acceptable excipient may have a particle diameter of more than, or equal to about: 30 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, or 400 μm. In some cases, at least a portion of particles of a pharmaceutically acceptable excipient may have a particle diameter of less than about: 30 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, or 400 μm. In some cases, the particles of a pharmaceutically acceptable excipient may range from about 50 μm to about 100 μm, which may be preferred when inhaled or administered intranasally for deposit on the oropharynx. In some instances, particle size as may comprise the diameter, the radius, or length of a particle. In some instances, particle size may be a measure of the mean, the median or the mode of a plurality of particles.

In some aspects, i) particles of an active ingredient (e.g. a serotonin receptor agonist) or a pharmaceutically acceptable salt thereof or ii) at least partially encapsulated particles comprising an active ingredient or a pharmaceutically acceptable salt thereof may have particle diameters ranging from about: 100 nm (nanometer) to about 500 nm, 300 nm to about 800 nm, 700 nm to about 1.2 μm, 1 μm to about 3 μm, 2 μm to about 4 μm, 3 μm to about 6 μm, 5 μm to about 8 μm, 6 μm to about 9 μm, 7 μm to about 10 μm, 8 μm to about 11 μm, 9 μm to about 13 μm, 10 μm to about 15 μm, 12 μm to about 20 μm, 14 μm to about 25 μm, or 18 μm to about 30 μm. In some cases, i) particles of an active ingredient or a pharmaceutically acceptable salt thereof or ii) at least partially encapsulated particles comprising an active ingredient or a pharmaceutically acceptable salt thereof may have a particle diameter of less than about: 30 nm, 50 nm, 60 nm, 70 nm, 80 nm, 90 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases i) particles of an active ingredient or a pharmaceutically acceptable salt thereof or ii) at least partially encapsulated particles comprising an active ingredient or a pharmaceutically acceptable salt thereof may have a particle diameter of more than about: 30 nm, 50 nm, 60 nm, 70 nm, 80 nm, 90 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, or 30 μm. In some cases, i) particles of an active ingredient or a pharmaceutically acceptable salt thereof or ii) at least partially encapsulated particles comprising an active ingredient or a pharmaceutically acceptable salt thereof may be in about 1 μm to about 5 μm, which may be preferred when inhaled or administered intranasally for absorption into lung alveoli.

In some aspects, a particles or compositions described herein may have a tap density of more than about: 0.1 grams/centimeter (g/cm³), 0.2 g/cm³, 0.3 g/cm³, 0.4 g/cm³ , 0.5 g/cm³, 0.6 g/cm³, 0.7 g/cm³, 0.8 g/cm³, 0.9 g/cm³, 1.0 g/cm³, 1.1 g/cm³ , or 1.2 g/cm³. In some aspects, a particles described herein may have a tap density of less than about: 0.1 g/cm³, 0.2 g/cm³, 0.3 g/cm³, 0.4 g/cm³, 0.5 g/cm³, 0.6 g/cm³, 0.7 g/cm³, 0.8 g/cm³, 0.9 g/cm³, 1.0 g/cm³, 1.1 g/cm³, or 1.2 g/cm³. In some cases, particles or compositions described herein may have a tap density of more than about 0.6 g/cm³ or 0.7 g/cm³. In some cases, particles or compositions described herein may have a tap density of about 0.6 g/cm³ or 0.7 g/cm³. In some cases, tap density of a powder may be the ratio of the mass of the powder to the volume occupied by the powder after it has been tapped for a defined period of time. In some cases, tap density may be a measure of the envelope mass density characterizing a particle. The envelope mass density of a particle of a statistically isotropic shape may be defined as the mass of the particle divided by the minimum sphere envelope volume within which it may be enclosed. Features which may contribute to low tap density include irregular surface texture, porous structure or a combination thereof. Tap density may be measured by using instruments known to those skilled in the art such as the Dual Platform Microprocessor Controlled Tap Density Tester (Vankel, N.C.) or a GeoPyc™ instrument (Micrometrics Instrument Corp., Norcross, Ga.).

In some aspects, particles of an active ingredient or a pharmaceutically acceptable salt thereof may be mixed in sizes. In some cases, the mixed sizes may change the release time of the drug. For example, particles with small sizes (e.g., about 1 μm to about 5 μm) may be readily absorbed into the blood stream while particles larger than about 10 μm may take longer to be absorbed into the blood stream. In some cases, particles with diameters of about 1 μm to about 10 μm may be inhaled into the lung while larger particles may be deposited onto the oropharynx. In some cases, particles with diameters of about 1 μm to about 5 μm may absorb faster than particles with diameters of about 7 μm to about 10 μm. In some aspects, the particles with sizes of about 7 μm to about 10 μm may be mixed with particles with sizes of about 1 μm to about 5 μm. In some aspects, the weight to weight ratio of the particles with diameters of about 7 μm to about 10 μm to the particles with sizes of about 1 μm to about 5 μm may range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some aspects, the weight to weight ratio of the particles with diameters of about 1 μm to about 5 μm to the particles with sizes of about 7 μm to about 10 μm may range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some aspects, the particles with larger sizes (about 10 μm to about 20 μm) may be mixed with particles with smaller sizes (about 1 μm to about 10 μm). In some aspects, the weight to weight ratio of the particles with larger sizes (about 10 μm to about 20 μm) to the particles with smaller sizes (about 1 μm to about 10 μm) may range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10. In some aspects, the weight to weight ratio of the particles with smaller sizes (about 1 μm to about 10 μm) to the particles with larger sizes (about 10 μm to about 20 μm) may range from about 1:1 to about 1:2, about 1:1 to about 1:3, about 1:1 to about 1:4, about 1:1 to about 1:5, about 1:1 to about 1:8, about 1:1 to about 1:10, about 1:2 to about 1:3, about 1:2 to about 1:4, about 1:2 to about 1:5, about 1:2 to about 1:8, about 1:2 to about 1:10, about 1:3 to about 1:4, about 1:3 to about 1:5, about 1:3 to about 1:8, about 1:3 to about 1:10, about 1:4 to about 1:5, about 1:4 to about 1:8, about 1:4 to about 1:10, about 1:5 to about 1:8, about 1:5 to about 1:10, or 1:8 to about 1:10.

In some aspects, active ingredient particles may be produced by spray drying. In some cases, encapsulated active ingredient particles may be produce by spray drying. In some instances, active ingredient particles may be produced by another method. In some instances, active ingredient particles may be produced by air-jet micronization, spiral milling, controlled precipitation, high-pressure homogenization, or cryo-milling. In some aspects, particle diameters may be measured by a particle analyzer using laser diffraction (LD), static light scattering, dynamic light scattering (DLS), or nanoparticle tracking analysis (NTA). In some aspects, particles that are not the pharmaceutically acceptable excipient, may have particle diameters ranging from about 1 μm to about 20 μm.

In some aspects, active ingredient particles may comprise a serotonin reception agonist drug such as a serotonin receptor agonist. In some instances, a serotonin receptor agonist may be blended with an excipient such as lactose or a salt thereof. In some instances, an excipient may comprise a lactose, a microcrystalline cellulose, a cellulose, a mannitol, a sorbitol, a starch, a starch glycolate, a hydroxypropyl methylcellulose, a hydroxypropyl methylcellulose acetate succinate, a cyclodextrin, a maltodextrin, a croscarmellose sodium, a corn starch, a carrageenan, a sorbitol, a maltitol, a glucose, a pharmaceutically acceptable salt of any of these, or any combination thereof.

In some cases, an encapsulated or unencapsulated serotonin receptor agonist, or a salt thereof may have a mean particle diameter of more than, less than, or equal to about: 30 nm, 50 nm, 60 nm, 70 nm, 80 nm, 90 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, or 20 μm. In some cases, an excipient or a salt thereof may have a mean particle diameter of more than, less than, or equal to about: 30 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm 140 μm, 145 μm 150 μm, 155 μm 160 μm, 165 μm 170 μm, 175 μm 180 μm, 185 μm 190 μm, 195 μm, 200 μm, 205 μm, 210 μm, 215 μm 220 μm, 225 μm 230 μm, 235 μm 240 μm, 245 μm, or 250 μm. In some instances, the shell of the microencapsulated particle comprises HPMCAS or HPMC. In some cases, microencapsulation a serotonin receptor agonist by HPMCAS may provide faster absorption in the lungs. For example, a serotonin receptor agonist may not be water soluble and microencapsulation with HPMCAS may provide increased absorption into the blood stream from the lungs. In some instances, microencapsulation may increase the solubility of an active ingredient. In some cases, a microencapsulated serotonin receptor agonist may be absorbed about: 10% to about 70%, 5% to about 10%, 5% to about 20%, 10% to about 30%, 15% to about 40%, 25% to about 40%, 35% to about 50%, 10 % to about 60%, 40% to about 90%, or 20% to about 50% faster than a serotonin receptor agonist that is not microencapsulated. In some cases, a microencapsulated serotonin receptor agonist, may be absorbed after inhalation into the blood stream in about: 5 seconds to about 30 seconds, 5 seconds to about 20 seconds, 10 seconds to about 20 seconds, 10 seconds to about 30 seconds, 10 seconds to about 60 seconds, 20 seconds to about 40 seconds, 30 second to about 60 seconds, 30 seconds to about 2 minutes, 1 minute to about 2 minutes, or 1 minute to 5 minutes.

In some instances, a serotonin receptor agonist, or a salt thereof, may be mixed with an excipient prior to adding to a capsule. In some cases, the mixing may comprise blending in a blender such as a V-type blender. In some cases, a serotonin receptor agonist may be mixed in a V-type blender with an excipient. A V-type blender may include a Patterson Kelly/PK Blender, a Gemco or a Ross blender. In some instances, blending may be high shear or low shear blending. In some cases, blending may be high speed or low speed blending. In some cases, the blending may distribute the serotonin agonist, or a salt thereof, and the excipient evenly. In some cases, the weight to weight ratio of the serotonin agonist, or a salt thereof, and the excipient may be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 55:1, 60:1, 65:1, 70:1, 75:1, 80:1, 85:1, 90:1, 95:1, or 100:1 (serotonin agonist to excipient). In some cases, the weight to weight ratio of the serotonin agonist, or a salt thereof, and the excipient may be about: 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:55, 1:60, 1:65, 1:70, 1:75, 1:80, 1:85, 1:90, 1:95, or 1:100 (serotonin agonist to excipient). In some aspects, an active ingredient or a pharmaceutically acceptable salt thereof (e.g., psilocybin or a pharmaceutically acceptable salt thereof) may comprise at least about: 1%, 2%, 3%, 4%, 5%, 6%, 7%, 8%, 9%, or 10% by weight of a pharmaceutical composition. The blending may not cause the excipient particle to be coated by the serotonin agonist particle or the salt thereof. In some instances, the serotonin receptor agonist such as psilocybin, or a pharmaceutically acceptable salt thereof, and an excipient may be administered via inhalation by a dry powder inhaler. In some cases, a dry powder inhaler does not comprise a propellent. In some cases, a dry powder inhaler does not comprise a chlorofluorocarbon, a hydrofluorocarbon, a fluorocarbon or any combination thereof as a propellent. In some cases, a dry powder inhaler is not pressurized. In some cases, an inhaler can comprise a propellent. In some instances, inhalation administration of serotonin agonist, or a salt thereof, and an excipient may produce about: 90%, 91%, 92%, 93%, 94%, 95%, 96%, 97%, 98%, 99% or 100% bioavailability of the serotonin receptor agonist. In some cases, the Tmax (e.g., the time required to reach the maximum concentration of a drug in the plasma) may be about: 5 min, 10 min, 15 min, 20 min, 25 min, 30 min, 35 min, 40 min, 45 min, 50 min, 55 min, 60 min, 65 min, 70 min, 75 min, 80 min, 85 min, 90 min, 95 min, 100 min, 105 min, 110 min, 115 min, 120 min, 130 min, 140 min, 150 min, 160 min, 170 min, 180 min, 190 min, 200 min, 210 min, 220 min, 230 min, 240 min, 250 min, 260 min, 270 min, 280 min, 290 min, or 300 min for a serotonin receptor agonist. In some cases, a composition can comprise a serotonin receptor agonist in an amount of more than, less than, or equal to about: 10 μg, 25 μg, 50 μg, 75 μg, 100 μg, 150 μg, 200 μg, 220 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg.

### Packaging of the Powdery Pharmaceutical Compositions

In some aspects, the pharmaceutical composition may be contained within a capsule, a capsule in capsule, a tablet, a gel, a gummy, a spray, an ointment, a paste, a jelly, an oil, a tincture, a lotion, a cream, a balm, a food, a drink, a liquid, a syrup, or any combination thereof.

In some aspects, the capsule may comprise a single-piece capsule, a two-piece capsule, a transparent capsule, a non-transparent capsule, an opaque capsule, a slow-release capsule, an extended-release capsule, a standard-release capsule, a rapid-release capsule, a quick-release capsule, a hard-shell capsule, a soft gel capsule, a gel capsule, a hard gelatin capsule, a soft gelatin capsule, an animal-based capsule, a vegetarian capsule, a polysaccharide capsule, a cellulose capsule, a mucopolysaccharide capsule, a tapioca capsule, a hydroxypropylmethyl cellulose (HPMC) capsule, a pullulan capsule, an enteric capsule, an uncoated capsule, a coated capsule, a capsule comprising titanium dioxide, fatty acids, waxes, shellac, plastics, plasticizers, glycerin, sorbitol, plant fibers, additives, preservatives, colorants, or any combination thereof.

In some aspects, the capsule can be sized according to the pharmaceutical composition requirements. In some aspects, the capsule size can be: 000, 00, 0, 1, 2, 3, or 4. In some aspects, the capsule size may be 000. In some aspects, the capsule size may be 00. In some aspects, the capsule size may be 0. In some aspects, the capsule size may be 1. In some aspects, the capsule size may be 2. In some aspects, the capsule size may be 3. In some aspects, the capsule size may be 4. In some aspects, the capsule capacity varies from about 0.21 ml to about 1.37 ml.

In some embodiments a capsule band can be added to a capsule. In some cases, capsule banding can be the process of sealing the capsule so that it may be filled with liquids, powders or other types of ingredients. In some instances, there can be a seal joint between the capsule cap and body that can require an additional band to be applied to prevent leakage of the drug outside the capsule. In some instances, it can provide a tamper resistant band that can reduce oxidation and minimizes any odor. In some cases, the banding can be applied with a banding machine that applies a thin layer of HPMC (hydroxypropyl methylcellulose) as the capsules pass over two rollers which apply the capsule banding material. In this process, the banding material can be heated and temperature controlled to make a smooth, liquid-tight band that join the capsule top and body. This can provide a visual tamper resistant barrier on the capsule.

In some aspects, the powdery pharmaceutical composition described herein when stored in a sealed container placed in a room at 25° C. and a room atmosphere having about 50 percent relative humidity, retains at least about: 80%, 90%, 95%, 96%, 97%, 98%, or 99% of the active ingredient or the salt thereof after 6 months, as measured by HPLC.

In some aspects, the pharmaceutical composition may be contained within a capsule. The capsule may be loaded with about 25% to about 75% (by volume) with a powdery pharmaceutical composition. In some cases, the capsule may be loaded with more than, less than, or equal to about: 30%, 31%, 32%, 33%, 34%, 35%, 36%, 37%, 38%, 39%, or 40% (by volume) of a pharmaceutical composition described herein. In some aspects, the capsule may be loaded with about 25% to about 30%, about 25% to about 40%, about 25% to about 50%, about 25% to about 60%, about 25% to about 65%, about 25% to about 70%, about 25% to about 75%, about 30% to about 40%, about 30% to about 50%, about 30% to about 60%, about 30% to about 65%, about 30% to about 70%, about 30% to about 75%, about 40% to about 50%, about 40% to about 60%, about 40% to about 65%, about 40% to about 70%, about 40% to about 75%, about 50% to about 60%, about 50% to about 65%, about 50% to about 70%, about 50% to about 75%, about 60% to about 65%, about 60% to about 70%, about 60% to about 75%, about 65% to about 70%, about 65% to about 75%, or 70% to about 75%, (by volume) of the powdery pharmaceutical composition.

In some aspects, the content of the capsule comprises less than about: 10%, 9%, 8%, 7%, 6%, 5%, 4%, 3%, 2%, or 1% water by weight. In some aspects, the content of the capsule comprises less than about 50%, about 40%, about 30%, about 25%, about 20%, about 10%, about 5%, or 1% water by weight. In some aspects, the total content of all gases in the capsule may be less than about: 10%, 9%, 8%, 7%, 6%, 5%, 4%, 3%, 2%, or 1% water by weight. In some aspects, the total content of all gases in the capsule may be less than about 50%, about 40%, about 30%, about 25%, about 20%, about 10%, about 5%, or 1% water by weight.

In some aspects, the capsule further comprises, in the volume not occupied by the powdery pharmaceutical composition, an inert gas. In some aspects, the inert gas comprises an elemental gas, a compound gas, a noble gas, helium, neon, argon, krypton, xenon, compounds of noble gas, purified argon, purified nitrogen, nitrogen, sulfur hexafluoride, or any combination thereof. In some aspects, the inert gas comprises nitrogen. In some cases, the inert gas within a capsule may comprise at least about: 75%, 80%, 85%, 90%, or 95% of the gas on a volume-to-volume basis.

In some aspects, the pharmaceutical composition may be contained within a capsule, wherein the capsule may be at least in part contained within a device which may be a drug delivery device, an inhalation drug delivery device, a diffuser, an inhaler, a metered dose inhaler, a dry powder inhaler, a soft mist inhaler, or any combination thereof. In some aspects, the device may be an inhaler. In some cases, a dry powder inhaler does not comprise a propellent. In some cases, a dry powder inhaler may not be pressurized. In some instances, a method of using a dry powder inhaler comprises breathing or inhaling an active ingredient or composition into the lungs. In some instances, a dry powder inhaler may be breath-activated, wherein when a subject breathes in through an inhaler, the inhaler releases particles (e.g., an active ingredient, excipient or both) which travel throughout the respiratory system. In some cases, a capsule may contain an active ingredient which may be pierced to release the particles prior to inhalation through a dry powder inhaler. In some instances, particle size and aerodynamics may affect travel throughout the respiratory system.

In some aspects, the pharmaceutical composition may be contained within a capsule, wherein the capsule may be at least in part contained within the device. In some aspects, the pharmaceutical composition may be contained within a capsule, wherein the capsule may be at least in part contained within the device, and wherein the device contains a sharp surface configured to puncture or slice the capsule. In some aspects, prior to administrating, the device may be actuated such that the sharp surface punctures or slices the capsule.

In some aspects, the pharmaceutical composition may be contained within a capsule, wherein the capsule may be at least in part contained within an inhaler, and wherein the inhaler contains a sharp surface configured to puncture or slice the capsule. In some aspects, the pharmaceutical composition may be contained within a capsule, wherein the capsule may be at least in part contained within an inhaler, and wherein the inhaler contains a sharp surface configured to puncture or slice the capsule, and wherein, prior to administrating, the inhaler may be actuated such that the sharp surface punctures or slices the capsule. In some aspects, the inhaler unit may be re-used via a process comprising replacing a spent capsule with a new capsule containing the powdery pharmaceutical composition. In some aspects, a component of the inhaler unit configured to at least in part hold the capsule may be temporarily at least partially separable from the inhaler unit. In some aspects, the capsule may be at least partially visible via an at least partially transparent material present in the inhaler unit.

Also disclosed herein are kits comprising the pharmaceutical composition contained at least in part in a packaging or a container. Also disclosed herein are methods of making kits comprising a pharmaceutical composition contained at least in part in a packaging or a container. In some cases, a container can comprise a plastic container, a metal container, a wood container, a glass container, or a combination thereof.

### Delivery and Administration of a Pharmaceutical Composition for the Treatment of Diseases

In some embodiments, a composition herein such as a pharmaceutical composition can be administered to treat a disease or condition. Also described herein are compositions for use in the treatment of diseases or conditions. In some aspects, the administration of a pharmaceutical composition or a second therapeutic may be administered orally, intra nasally, intra ocular, anally, by injection, intravenously, intramuscularly, subcutaneously, intraperitoneally, transdermally, or any combination thereof.

In some aspects, the administration of the pharmaceutical composition may be by inhalation. In some aspects, inhalation may be oral inhalation, intra nasal administration, or any combination thereof. In some aspects, the powdery pharmaceutical composition may be inhaled into human lungs. In some cases, inhaled may be inhalation through the mouth, for example with a dry powdered inhaler. In some cases, at least a portion of the excipient may deposit on the oropharynx. In some aspects, the powdery pharmaceutical composition, when inhaled into the lungs, provides a time to peak plasma concentration (Tmax) of the active ingredient or the salt thereof. The time to peak plasma concentration (Tmax) of the active ingredient or the salt thereof may range from about 1 minute to about one hour. In some aspects, the time to peak plasma concentration (Tmax) of the active ingredient or the salt thereof may range from about 1 minute to about ten minutes.

In some aspects, administering may be by oral ingestion, topical application, or inhalation. In some aspects, administering may comprise oral ingestion and the oral ingestion may comprise oral ingestion of a food, a liquid, a gel, a capsule, or any combination thereof. In some aspects, administering may comprise topical application and the topical application may comprise topical application of a lotion, a tincture, a balm, a cream, an oil, a gel, a butter, a liquid, a spray, an ointment, a paste, a jelly, or any combination thereof. In some aspects, administering may comprise inhalation and the inhalation may comprise inhalation by a diffuser, an inhaler, a nebulizer, or any combination thereof. In some aspects, administering may comprise inhalation and the inhalation may comprise inhalation by a diffuser or inhaler. In some aspects, administering may comprise inhalation and the inhalation may comprise inhalation by a nebulizer. In some aspects, administering may be performed at least about: 1 time per day, 2 times per day, 3 times per day, 4 times per day, 5 times per day, 6 times per day or more than 6 times per day. In some cases, administering may be performed daily, weekly, monthly, or as needed. In some aspects, administering may be conducted one, twice, three, or four times per day. In some cases, administration may be provided by a subject (e.g., the patient), a health care provider, or both.

In some aspects, administering may be performed for about: 1 day to about 8 days, 1 week to about 5 weeks, 1 month to about 12 months, 1 year to about 3 years, 3 years to about 10 years, 10 years to about 50 years, 25 years to about 100 years, or 50 years to about 130 years. In some aspects, the composition may be administered as needed, or for: one day, two days, three days, four days, five days, six days, a week, two weeks, three weeks, a month, two months, three months, four months, five months, six months, seven months, eight months, nine months, ten months, eleven months, a year, or chronically.

In some aspects, prior to treating, a subject may have been diagnosed with a disease. In some aspects, during treatment, a subject is diagnosed with a disease. In some aspects, a subject may not have been diagnosed with a disease. In some aspects, the subject may be a human, a man, a woman, an individual over 18 years of age, an individual under 18 years of age, or any combination thereof. In some aspects, a subject may be from about 1 day to about 10 months old, from about 9 months to about 24 months old, from about 1 year to about 8 years old, from about 5 years to about 25 years old, from about 20 years to about 50 years old, from about 40 years to about 80 years old, or from about 50 years to about 130 years old.

In some aspects, a method may further comprise diagnosing a subject as having the disease. In some aspects, a diagnosing may comprise employing an in vitro diagnostic. In some aspects, the in vitro diagnostic may be a companion diagnostic. In some aspects, a diagnosis may comprise a physical examination, a radiological image, a blood test, an antibody test, or any combination thereof. In some aspects, a diagnosis may comprise a radiological image and the radiological image may comprise: a computed tomography (CT) image, an X-Ray image, a magnetic resonance image (MRI), an ultrasound image, or any combination thereof.

In some aspects, the composition may be administered so that the active ingredient or the pharmaceutically acceptable salt thereof in the unit dose ranges from about: 500 μg (micrograms) to about 1000 mg, 10 μg to about 50 μg, 40 μg to about 90 μg, 80 μg to about 12 0μg, 100 μg to about 150 μg, 140 μg to about 190 μg, 150 μg to about 220 μg, 200 μg to about 25 0μg, 240 μg to about 300 μg, 290 μg to about 350 μg, 340 μg to about 410 μg, 400 μg to about 450 μg, 440 μg to about 500 μg, 500 μg to about 700 μg, 600 μg to about 900 μg, 800 μg to about 1 mg, 1 mg to about 5 mg, 1 mg to about 10 mg, 5 mg to about 15 mg, 12 mg to about 25 mg, 20 mg to about 50 mg, 40 mg to about 80 mg, 70 mg to about 100 mg, 90 mg to about 150 mg, 125 mg to about 250 mg, 200 mg to about 500 mg, 400 mg to about 750 mg, 700 mg to about 900 mg, or from about 850 mg to about 1000 mg. In some cases, the unit dose range may be more than, or equal to about: 10 μg, 25 μg, 50 μg, 75 μg, 100 μg, 150 μg, 200 μg, 220 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg. In some cases, the unit dose range may be less than about: 10 μg, 25 μg, 50μg, 75 μg, 100 μg, 150 μg, 200 μg, 220 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg. In some cases, serotonin agonist or a salt thereof may be administered in a unit dose form of about 0.22 mg. In some cases, serotonin agonist or a salt thereof may be administered in a unit dose form of about 0.10, 0.20, 0.30, 0.40 or 0.50 mg.

### Treatment of Diseases and Conditions

Also disclosed herein are methods of treating a disease comprising treating the disease or condition by administering a therapeutically effective amount of a powdery pharmaceutical composition. In some cases, the use of a therapeutically effective amount of a powdery pharmaceutical composition to treat a disease or condition is disclosed herein. In some cases, the use of a composition herein in the manufacture of a medicament for the treatment of a disease or condition is disclosed herein. Also disclosed herein are methods of treating a disease comprising treating the disease or condition by administering, via inhalation, a therapeutically effective amount of the powdery pharmaceutical composition. In some aspects, the disease or condition may comprise an immune condition, a cardiac disorder, a mood disorder, a psychiatric disorder, a cancer, an infection, or any combination thereof.

In some cases, a powdery pharmaceutical formulation disclosed herein may be administered to treat an immune disorder such as allergies, asthma, anaphylaxis, an autoimmune disease, a disease or condition characterized by inefficient immune response, or any combination thereof. In some cases, a powdery pharmaceutical formulation disclosed herein may be administered to treat a circulatory system disorder such as hypertension, hypotension, cardiac arrest, a stroke, a heart failure, a peripheral arterial disease, an atherosclerosis, a dysrhythmia, an arrhythmia, or any combination thereof.

In some cases, a powdery pharmaceutical formulation disclosed herein may be administered to treat a post-traumatic stress disorder, a depression, suicidal tendencies, a generalized anxiety disorder, a panic disorder (e.g., a panic attack), a phobia, a social anxiety disorder, an attention deficit hyperactivity disorder, agoraphobia, a personality disorder, or any combination thereof. In some cases, a depression can comprise a major depression, a persistent depressive disorder, a bipolar disorder (e.g., bipolar 1, bipolar 2, cyclothymic disorder), a seasonal affective disorder, a perinatal depression, a Premenstrual dysphoric disorder, or any combination thereof. In some cases, a personality disorder can comprise an antisocial personality disorder, an avoidant personality disorder, a borderline personality disorder, a dependent personality disorder, a histrionic personality disorder, a narcissistic personality disorder, an obsessive-compulsive personality disorder, a paranoid personality disorder, or any combination thereof. In some cases, a formulation herein can be used to treat a psychiatric disorder. In some cases, a psychiatric disorder may comprise autism spectrum disorder, a communication disorder, a bipolar disorder, an anxiety disorder, a phobia, a stress-related disorder, a dissociate disorder, a somatic symptom disorder, an eating disorder, a sleep disorder, a disruptive disorder, a depressive disorder, a substance related disorder, a mental addiction, a neurocognitive disorder (such as memory loss, senility, Alzheimer's disease and the like), a lack of desired neurogenesis, a schizophrenia, an obsessive-compulsive disorder, a personality disorder, or any combination thereof. In some cases, a stress-related disorder can comprise a reactive attachment disorder, a disinhibited social engagement disorder, an acute stress disorder, an adjustment disorder, a post-traumatic stress disorder, or any combination thereof. In some cases, an anxiety disorder can comprise generalized anxiety, agoraphobia, a panic disorder, a social anxiety disorder, a separation anxiety disorder, or any combination thereof. In some cases, an eating disorder can comprise an anorexia nervosa, a bulimia nervosa, a binge-eating disorder, an avoidant restrictive food intake disorder or any combination thereof. In some cases, a schizophrenia can comprise a paranoid schizophrenia, a hebephrenic schizophrenia, a catatonic schizophrenia, an undifferentiated schizophrenia, a residential schizophrenia, a simple schizophrenia, a cenesthopathic schizophrenia, an unspecified schizophrenia, or any combination thereof. In some cases, a communication disorder can comprise a speech disorder, a language disorder, a hearing disorder, or a central auditory processing disorder. In some cases, a sleep disorder can comprise an insomnia, a sleep apnea, a narcolepsy, a restless legs syndrome, a parasomnia, an excessive sleepiness, a shift word disorder, a non-24 hour sleep wake disorder, or any combination thereof.

In some cases, a powdery pharmaceutical formulation disclosed herein may be administered to treat an addiction or withdrawal symptoms associated with the addiction. Examples may include opioid addiction, cannabis addiction, benzodiazepine addiction, alcohol addiction, barbiturate addiction, cocaine addiction, amphetamine addiction, methamphetamine addiction, nicotine/tobacco addiction, sex addiction, gambling addiction, food addiction, or any combination thereof.

In some cases, a powdery pharmaceutical formulation disclosed herein may be administered to treat an infection. Such an infection may comprise a bacterial infection, a viral infection, a parasitic infection or a fungal infection.

In some cases, a powdery pharmaceutical formulation disclosed herein may be administered to treat a cancer such as: a melanoma, a hepatocellular carcinoma, a breast cancer, a lung cancer, a non-small lung cancer, a peritoneal cancer, a prostate cancer, a bladder cancer, an ovarian cancer, a leukemia, a lymphoma, a renal cell carcinoma, a pancreatic cancer, an epithelial carcinoma, a gastric/GE junction adenocarcinoma, a cervical cancer, a colon carcinoma, a colorectal cancer, a duodenal cancer, a pancreatic adenocarcinoma, an adenoid cystic, a sarcoma, a mesothelioma, a glioblastoma multiforme, a astrocytoma, a multiple myeloma, a prostate carcinoma, a hepatocellular carcinoma, a cholangiocarcinoma, a pancreatic adenocarcinoma, a head and neck squamous cell carcinoma, a cervical squamous-cell carcinoma, an osteosarcoma, an epithelial ovarian carcinoma, an acute lymphoblastic lymphoma, a myeloproliferative neoplasm, any other malignant condition, or any combination thereof.

In some cases, a powdery pharmaceutical formulation disclosed herein may be administered to treat a side effect from chemotherapy such as a peripheral neuropathy, a cognitive impairment, a nausea, a weight loss, an increase in bruising, a fatigue, an infection, a mouth pain, a urinary issue, a libido loss, or any combination thereof.

In some cases, a powdery pharmaceutical formulation disclosed herein may be administered to treat a weight disorder. In some cases, a weight disorder (e.g., an eating disorder) can comprise an obesity, an increased appetite, a glucose intolerance, an anorexia nervosa, a binge eating disorder, a pica disorder, a rumination disorder, or any combination thereof. In some cases, obesity can comprise an overweight, a class 1 obesity (e.g. a body mass index (BMI) of 30 to <35), a class 2 obesity (e.g., a BMI of 35 to <40), or a class 3 obesity (a BMI of 40 or higher) . In some cases, a powdery pharmaceutical formulation disclosed herein may be administered to treat a diabetes or prediabetes. In some cases, a diabetes can comprise type 1 diabetes, type 2 diabetes, or gestational diabetes.

In some cases, a powdery pharmaceutical formulation disclosed herein may be administered to treat a seizure, such as an epileptic seizure. In some cases, a seizure can comprise a generalized seizure, an absence seizure, a tonic-clonic seizure, a focal seizure, a simple focal seizure, a complex focal seizure, a secondary generalized seizure, or any combination thereof. In some cases, a powdery pharmaceutical formulation disclosed herein may be administered to treat a croup, a shock or both. In some cases, a powdery pharmaceutical formulation disclosed herein may be administered to increase an exercise performance, an athletic performance, or both.

In some aspects, a method may further comprise administering a second therapy to the subject. In some aspects, an additional therapy may comprise a monoclonal antibody such as an eptinezumab, a fremanezumab, an erenumab, a galcanezumab, a biologically active fragment of any of these, a salt of any of these, or any combination thereof. In some aspects, an additional therapy may comprise a nonsteroidal anti-inflammatory drug and the nonsteroidal anti-inflammatory drug may comprise, an aspirin, a naproxen, an ibuprofen, a diclofenac, a celecoxib, a mefenamic acid, an etoricoxib, an indomethacin, a salt of any of these, or any combination thereof. In some aspects, a composition may comprise an excipient, a diluent, a carrier, or any combination thereof. In some aspects, other pain non-opioid pain relievers may be used such as acetaminophens. In other aspects, opioid pain relievers may be used such as hydrocodone, an oxycodone, a morphine, a codeine, a hydromorphone, an oxymorphone, a salt of any of these, or any combination thereof. In still other aspects, a composition may comprise a caffeine, a theobromine, a salt of any of these, or any combination thereof.

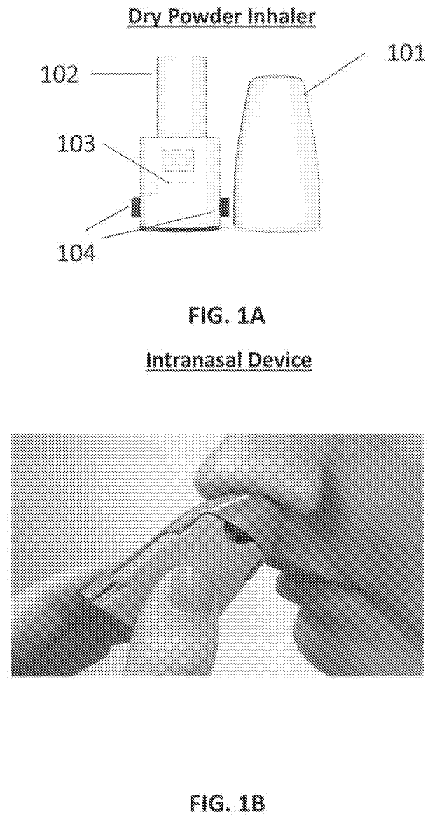

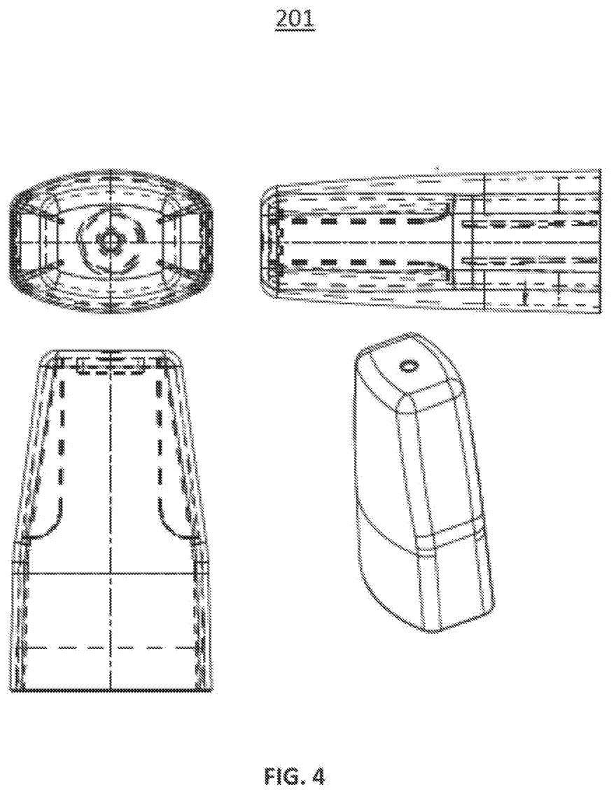

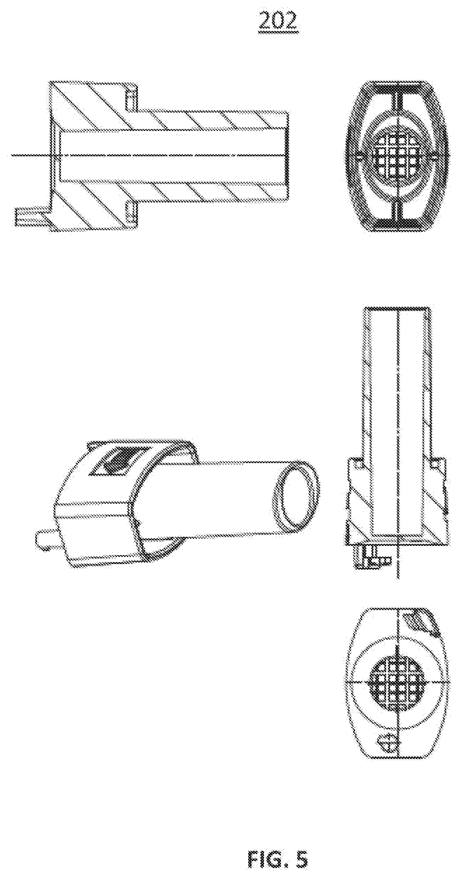

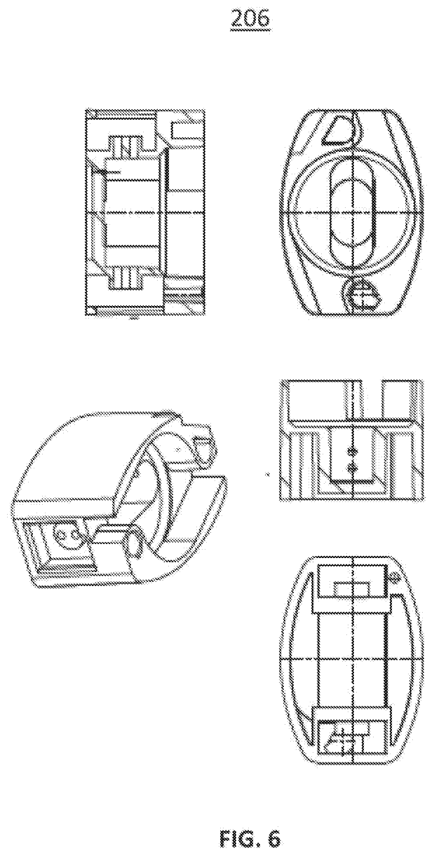

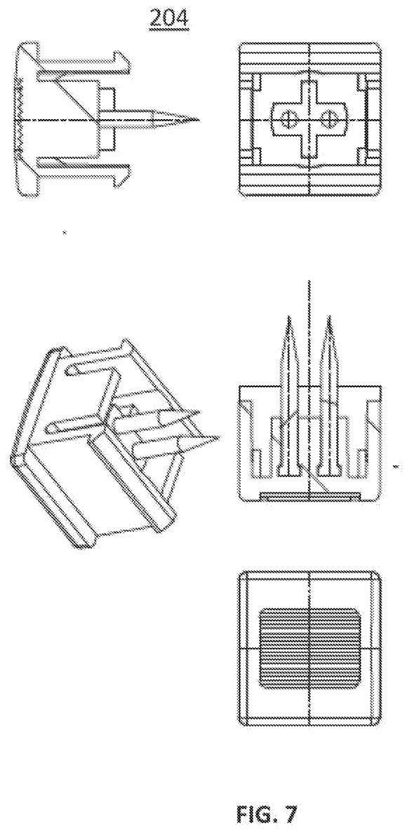

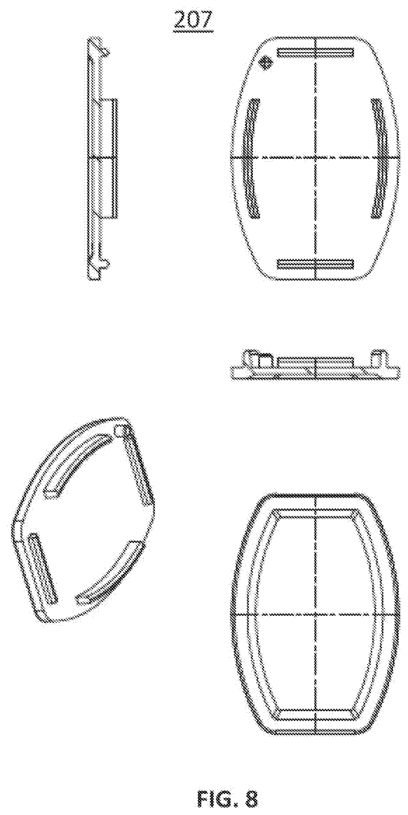

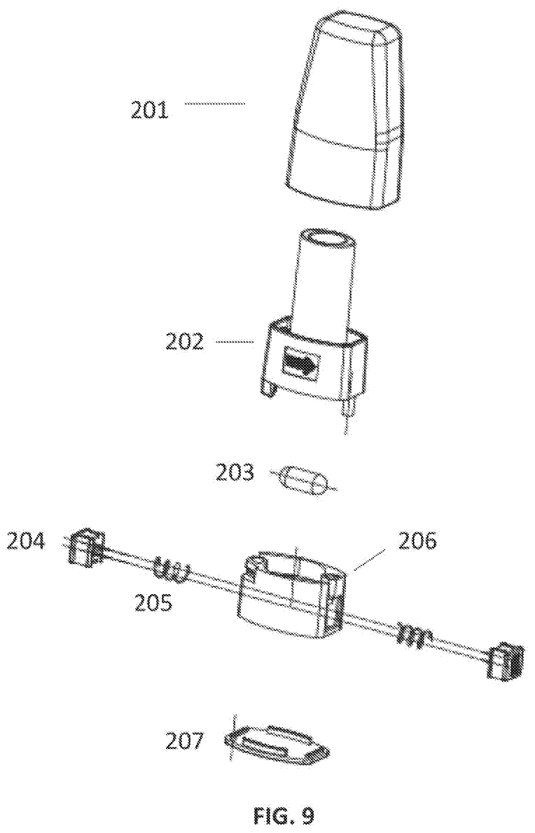

Referring to FIG. 1, FIG. 1A shows a dry powder inhaler device for delivery of powdery pharmaceutical compositions to the lung alveolar. The inhaler device may comprise a protective cap shown in FIG. 4, a rotatable top comprising a mouthpiece shown in FIG. 5, a lower base chamber receptacle for placing a pharmaceutical capsule shown in FIG. 6, a lateral button for mechanically piercing a capsule with a sharp surface while inside the chamber show in FIG. 7, and a chamber aerially connected to the mouthpiece permitting inhalation of capsule contents. The dry powder inhaler device may comprise a base plate as shown in FIG. 8. FIG. 9 shows a dry powder inhaler device with a protective cap, a rotatable comprising a mouthpiece, a lower base chamber for piercing a pill and a base plate.

### Numbered Embodiments

A number of compositions, kits, and methods are disclosed herein. Specific exemplary embodiments of these compositions, kits, and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. A powdery pharmaceutical composition, comprising: a) particles of a pharmaceutically acceptable excipient; and b) a plurality of spray dried particles, wherein each particle of the plurality of spray dried particles is substantially encapsulated in a coating material and wherein the plurality of spray dried particles substantially encapsulated in the coating material comprise: an agonist selected from the group consisting of: a substituted tryptamine or a pharmaceutically acceptable salt thereof, an amide of lysergic acid or a pharmaceutical acceptable salt thereof, and a N-Methyl-D-aspartate receptor agonist or a pharmaceutical acceptable salt thereof; and wherein within the plurality of spray dried particles substantially encapsulated in the coating material at least a portion of the spray dried particles substantially encapsulated in the coating material individually have a particle diameter ranging from about 1 micrometer to about 10 micrometers, as measured by a particle analyzer using laser diffraction; and wherein the coating material comprises trehalose, a hydroxypropyl methylcellulose (HPMC), a fumaryl diketopiperazine (FDKP), 1,2-distearoyl-sn-glycero-3-phosphocholine, a hydroxypropyl methylcellulose acetate succinate (HPMCAS), a povidone, a copovidone or any combination thereof.

Embodiment 2. The powdery pharmaceutical composition of embodiment 1, wherein the spray dried particles comprise the substituted tryptamine.

Embodiment 3. The powdery pharmaceutical composition of embodiments 2, wherein the substituted tryptamine is a psilocin or a salt thereof.

Embodiment 4. The powdery pharmaceutical composition of embodiment 2, wherein the substituted tryptamine is a psilocybin or a salt thereof.

Embodiment 5. The powdery pharmaceutical composition of embodiment 2, wherein the substituted tryptamine is an ibogaine or a salt thereof.

Embodiment 6. The powdery pharmaceutical composition of embodiment 1, wherein the spray dried particles comprise the N-Methyl-D-aspartate receptor agonist.

Embodiment 7. The powdery pharmaceutical composition of embodiment 6, wherein the N-Methyl-D-aspartate receptor agonist is a ketamine or a salt thereof.

Embodiment 8. The powdery pharmaceutical composition of any one of embodiments 1-7, wherein the powdery pharmaceutical composition is for inhaled use or for intranasal use.

Embodiment 9. The powdery pharmaceutical composition of any one of embodiments 1-8, wherein the pharmaceutical composition is in unit dose form.

Embodiment 10. The powdery pharmaceutical composition of any one of embodiments 1-9, wherein at least a portion of the particles of the pharmaceutically acceptable excipient individually have a particle diameter ranging from about 1 micrometers to about 200 micrometers, or about 1 micrometer to about 10 micrometers, as measured by a particle analyzer using laser diffraction.

Embodiment 11. The powdery pharmaceutical composition of any one of embodiments 1-10, wherein the particles are admixed into a substantially homologous mixture.

Embodiment 12. The powdery pharmaceutical composition of any one of embodiments 1-11, which is contained within a capsule.

Embodiment 13. The powdery pharmaceutical composition of embodiment 12, wherein the capsule is about one quarter to about one half, by volume, filled with the powdery pharmaceutical composition.

Embodiment 14. The powdery pharmaceutical composition of any one of embodiments 1-13, wherein a weight-to-weight ratio of: a) the particles of the pharmaceutically acceptable excipient and b) the particles comprising the agonist or a pharmaceutically acceptable salt thereof, substantially encapsulated in a coating material, ranges from about 1:1 (w/w) to about 10000:1 (w/w).

Embodiment 15. The powdery pharmaceutical composition of embodiment 14, wherein the weight-to-weight ratio of: a) the particles of the pharmaceutically acceptable excipient and b) the particles of the agonist or the pharmaceutically acceptable salt thereof, ranges from about 1:1 (w/w) to about 10:1 (w/w).

Embodiment 16. The powdery pharmaceutical composition of any one of embodiments 12-15, wherein the portion of the capsule not containing the powdery pharmaceutical composition comprises a gas that at least partially comprises an inert gas.

Embodiment 17. The powdery pharmaceutical composition of embodiment 16, wherein the inert gas comprises nitrogen, carbon dioxide, helium, or any combination thereof.

Embodiment 18. The powdery pharmaceutical composition of embodiment 16 or 17, wherein the inert gas comprises at least about: 80%, 85%, 90%, or 95% of the gas on a volume-to-volume basis.

Embodiment 19. The powdery pharmaceutical composition of any one of embodiments 16-18, wherein i) the powdery pharmaceutical composition within the capsule, ii) the gas within the capsule, or iii) any combination thereof comprises less than about 10% water by weight based on the weight of the powdery pharmaceutical composition or a total content of all gases in the capsule is less than about 10% water by weight within: the powdery pharmaceutical composition within the capsule, the gas within the capsule, or any combination thereof.

Embodiment 20. The powdery pharmaceutical composition of any one of embodiments 12-19, wherein the capsule comprises a hydroxypropylmethyl cellulose (HPMC) capsule.

Embodiment 21. The powdery pharmaceutical composition of any one of embodiments 12-20, wherein the capsule is size: 000, 00, 0, 1, 2, 3, or 4.

Embodiment 22. The powdery pharmaceutical composition of embodiment 21, comprising the capsule, wherein the capsule is size 3.

Embodiment 23. The powdery pharmaceutical composition of any one of embodiments 1-22, contained within an inhaler unit.

Embodiment 24. The powdery pharmaceutical composition of any one of embodiments 12-23, wherein the capsule is contained in an inhaler unit.

Embodiment 25. The powdery pharmaceutical composition of any one of embodiments 1-24, wherein the pharmaceutically acceptable excipient comprises a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof.

Embodiment 26. The powdery pharmaceutical composition of embodiment 25, wherein the pharmaceutically acceptable excipient or pharmaceutically acceptable salt thereof comprises the carbohydrate or the pharmaceutically acceptable salt thereof, and wherein the carbohydrate or the pharmaceutically acceptable salt thereof comprises lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, a pharmaceutically acceptable salt of any of these, or any combination thereof.

Embodiment 27. The powdery pharmaceutical composition of embodiment 26, wherein the pharmaceutically acceptable excipient or the pharmaceutically acceptable salt thereof comprises lactose or a pharmaceutically acceptable salt thereof.

Embodiment 28. The powdery pharmaceutical composition of embodiment 27, comprising the lactose or the pharmaceutically acceptable salt thereof, which comprises milled lactose, sieved lactose, micronized lactose, spray dried lactose, at least substantially anhydrous lactose, monohy drate lactose, a pharmaceutically acceptable salt thereof, or any combination thereof.

Embodiment 29. The powdery pharmaceutical composition of any one of embodiments 1-28, wherein the powdery pharmaceutical composition described herein when stored in a sealed container placed in a room at 25° C. and a room atmosphere having about 50 percent relative humidity, retains at least about: 90% of the agonist thereof after 6 months, as measured by HPLC.

Embodiment 30. The powdery pharmaceutical composition of any one of embodiments 1-29, wherein the agonist is present in an amount ranging from about 0.001 mg to about 20 mg.

Embodiment 31. The powdery pharmaceutical composition of any one of embodiments 1-30, wherein the agonist is in the form of a pharmaceutically acceptable salt thereof and is a hydrochloride salt, a bitartrate salt or a borate salt.

Embodiment 32. The powdery pharmaceutical composition of any one of embodiments 1-31, wherein the particles comprising the agonist comprise a median diameter of less than 5 μm.

Embodiment 33. The powdery pharmaceutical composition of any one of embodiments 1-32, wherein the particles comprising the agonist or the pharmaceutically acceptable salt thereof comprise a median diameter of less than about: 6 μm, 7 μm, 8 μm, 9 μm or 10 μm.

Embodiment 34. A kit comprising the powdery pharmaceutical composition of any one of embodiments 1-33 contained at least in part in a container.

Embodiment 35. A method of treating or preventing a disease or condition in a subject in need thereof, comprising treating or preventing the disease or condition by administering, via inhalation, a first therapeutic comprising a therapeutically effective amount of the powdery pharmaceutical composition of any one of embodiments 1-33 to the subject in need thereof.

Embodiment 36. The method of embodiment 35, wherein the administering is conducted one, twice, three, or four times per day.

Embodiment 37. The method of embodiment 35 or 36, wherein the disease or condition is selected from the group consisting of: a migraine headache, a tension headache and a cluster headache.

Embodiment 38. The method of embodiment 35 or 36, wherein the disease or condition is selected from the group consisting of: a post-traumatic stress disorder, a depression, a generalized anxiety disorder, a panic disorder, a phobia, a social anxiety disorder, attention deficit hyperactivity disorder, an agoraphobia and a personality disorder.

Embodiment 39. The method of embodiment 35 or 36, wherein the disease or condition is selected from the group consisting of: alcohol addiction, cocaine addiction, methamphetamine addiction, opioid addiction, nicotine addiction, and gambling addiction.

Embodiment 40. The method of any one of embodiments 35-39, wherein the powdery pharmaceutical composition is administered as needed, or for about: one day, two days, three days, four days, five days, six days, a week, two weeks, three weeks, a month, two months, three months, four months, five months, six months, seven months, eight months, nine months, ten months, eleven months, a year, or chronically.

Embodiment 41. The method of any one of embodiments 35-40, wherein a second therapeutic or pharmaceutically acceptable salt thereof is administered.

Embodiment 42. The method of embodiment 41, wherein the second therapeutic or a pharmaceutically acceptable salt thereof is administered concurrently or consecutively.

Embodiment 43. The method of embodiment 41 or 42, wherein the second therapeutic or the pharmaceutically acceptable salt thereof is comprised in the powdery pharmaceutical formulation.

Embodiment 44. The method of embodiment 43, wherein when the first therapeutic comprises the substituted tryptamine, the second therapeutic comprises the N-Methyl-D-aspartate receptor agonist.

Embodiment 45. The method of embodiment 43, wherein when the first therapeutic comprises the amide of lysergic acid, the second therapeutic comprises the N-Methyl-D-aspartate receptor agonist.

Embodiment 46. The method of any one of embodiments 35-45 further comprising administering caffeine, a non-steroidal anti-inflammatory drug, an anti-calcitonin gene-related peptide monoclonal antibody or a combination thereof to the subject.

Embodiment 47. The method of any one of embodiments 35-46, wherein the subject is diagnosed with the disease or condition.

Embodiment 48. The method of embodiment 47, wherein the diagnosing comprises employing an in vitro diagnostic.

Embodiment 49. The method of embodiment 48, wherein the in vitro diagnostic is a companion diagnostic.

Embodiment 50. The method of any one of embodiments 35-49, wherein the powdery pharmaceutical composition is contained within a capsule, wherein the capsule is at least in part contained within an inhaler, and wherein the inhaler contains a sharp surface configured to puncture or slice the capsule, and wherein, prior to administrating, the inhaler is actuated such that the sharp surface punctures or slices the capsule.

Embodiment 51. The method of any one of embodiments 35-50, wherein the inhalation is oral inhalation, intra nasal administration, or any combination thereof.

Embodiment 52. A method for making a powdery pharmaceutical composition, comprising contacting in a solution: i) an agonist comprising a substituted tryptamine, or a pharmaceutically acceptable salt thereof, an amide of lysergic acid, or a pharmaceutically acceptable salt thereof, a N-Methyl-D-aspartate receptor agonist or a pharmaceutical acceptable salt thereof; ii) a coating material comprising trehalose, a fumaryl diketopiperazine (FDKP), 1,2-distearoyl-sn-glycero-3-phosphocholine, a hydroxypropyl methylcellulose (HPMC), a hydroxypropyl methylcellulose acetate succinate (HPMCAS), a povidone, a copovidone or any combination thereof, and iii) a solvent; wherein the solution is spray dried to form substantially encapsulated particles.

Embodiment 53. The powdery pharmaceutical composition of embodiment 52, wherein the spray drying comprises: a) atomizing liquid droplets comprising the agonist or the pharmaceutically acceptable salt thereof, the coating material, and the solvent, b) drying the droplets to form the substantially encapsulated particles, wherein the substantially encapsulated particles comprise the agonist or the pharmaceutically acceptable salt thereof substantially encapsulated by the coating material and b) recovering the substantially encapsulated particles.

Embodiment 54. A powdery pharmaceutical composition, comprising: a) particles of a pharmaceutically acceptable excipient; and b) a plurality of spray dried particles, wherein each particle of the plurality of spray dried particles is substantially encapsulated in a coating material and wherein the plurality of spray dried particles substantially encapsulated in the coating material comprise N-lactoyl-phenylalanine or a pharmaceutically acceptable salt thereof; wherein within the plurality of spray dried particles substantially encapsulated in the coating material at least a portion of the spray dried particles substantially encapsulated in the coating material individually have a particle diameter ranging from about 1 micrometer to about 10 micrometers, as measured by a particle analyzer using laser diffraction; and wherein the coating material comprises trehalose, a hydroxypropyl methylcellulose (HPMC), a fumaryl diketopiperazine (FDKP), 1,2-distearoyl-sn-glycero-3-phosphocholine, a hydroxypropyl methylcellulose acetate succinate (HPMCAS), a povidone, a copovidone or any combination thereof.

Embodiment 55. A method of treating or preventing a disease or condition in a subject in need thereof, comprising treating or preventing the disease or condition by administering, via inhalation, a first therapeutic comprising a therapeutically effective amount of the powdery pharmaceutical composition of embodiment 54 to the subject in need thereof.

Embodiment 56. The method of embodiment 55, wherein the administering is conducted one, twice, three, or four times per day.

Embodiment 57. The method of embodiment 55 or 56, wherein the disease or condition is selected from the group consisting of: obesity, increased appetite, or glucose intolerance.

### Numbered Embodiments Overview

Certain aspects of the disclosure herein pertain to a powdery pharmaceutical composition, comprising: a) particles of a pharmaceutically acceptable excipient; and b) a plurality of spray dried particles, wherein each particle of the plurality of spray dried particles is substantially encapsulated in a coating material and wherein the plurality of spray dried particles substantially encapsulated in the coating material comprise: an agonist selected from the group consisting of: a substituted tryptamine or a pharmaceutically acceptable salt thereof, an amide of lysergic acid or a pharmaceutical acceptable salt thereof, and a N-Methyl-D-aspartate receptor agonist or a pharmaceutical acceptable salt thereof; and wherein within the plurality of spray dried particles substantially encapsulated in the coating material at least a portion of the spray dried particles substantially encapsulated in the coating material individually have a particle diameter ranging from about 1 micrometer to about 10 micrometers, as measured by a particle analyzer using laser diffraction; and wherein the coating material comprises a hydroxypropyl methylcellulose (HPMC), a fumaryl diketopiperazine (FDKP), 1,2-distearoyl-sn-glycero-3-phosphocholine, a hydroxypropyl methylcellulose acetate succinate (HPMCAS), a povidone, a copovidone or any combination thereof.

In certain cases, the powdery pharmaceutical composition comprises the substituted tryptamine. In certain cases, the substituted tryptamine is psilocin. In other cases, the substituted tryptamine is psilocybin. In other cases, the substituted tryptamine is an amide of lysergic acid. In other cases, the substituted tryptamine is a triptan.

In some cases, concerning the powdery pharmaceutical composition, spray dried particles comprise the N-Methyl-D-aspartate receptor agonist. In some cases, N-Methyl-D-aspartate receptor agonist is ketamine.

In certain instances, the powdery pharmaceutical composition of as described herein is for inhaled use or for intranasal use. In some instances, the pharmaceutical composition as described herein. In some instances, the pharmaceutical formulation is for gastrointestinal delivery.

In some cases, the powdery pharmaceutical composition herein is in unit dose form. In some cases the powdery pharmaceutical composition as described above has at least a portion of the particles of the pharmaceutically acceptable excipient individually have a particle diameter ranging from about 1 micrometers to about 200 micrometers, or about 1 micrometer to about 10 micrometers, as measured by a particle analyzer using laser diffraction. In certain cases, the pharmaceutical composition as described above comprises

In some instances, the powdery pharmaceutical composition as described above comprises particles that are admixed into a substantially homologous mixture. In certain cases, the pharmaceutical composition is contained in a capsule. In certain cases, the capsule is about one quarter to about one half, by volume, filled with the powdery pharmaceutical composition. In certain cases, the pharmaceutical composition as described above comprises a weight-to-weight ratio of: a) the particles of the pharmaceutically acceptable excipient and b) the particles comprising the agonist or a pharmaceutically acceptable salt thereof, substantially encapsulated in a coating material, ranges from about 1:1 (w/w) to about 10000:1 (w/w). In certain cases, the weight-to-weight ratio of: a) the particles of the pharmaceutically acceptable excipient and b) the particles of the agonist or the pharmaceutically acceptable salt thereof, ranges from about 1:1 (w/w) to about 10:1 (w/w). Still further, in certain cases, the portion of the capsule not containing the powdery pharmaceutical composition comprises a gas that at least partially comprises an inert gas. The inert gas may comprise nitrogen, carbon dioxide, helium, or any combination thereof. In certain cases, the inert gas comprises at least about: 80%, 85%, 90%, or 95% of the gas on a volume-to-volume basis.

In certain cases, regarding the powdery pharmaceutical composition as described above, the composition comprises less than about 10% water by weight based on the weight of the powdery pharmaceutical composition or a total content of all gases in the capsule is less than about 10% water by weight within: the powdery pharmaceutical composition within the capsule, the gas within the capsule, or any combination thereof.

In certain instances, the pharmaceutical composition is in capsule form and comprises a hydroxypropylmethyl cellulose (HPMC) capsule. In certain instances, the powdery the capsule is size: 000, 00, 0, 1, 2, 3, or 4. In specific instances, the capsule size is size 3.

In certain instances, the powdery pharmaceutical composition as described above is contained within an inhaler unit. In certain cases, the capsule is contained in an inhaler unit. In certain cases, the pharmaceutically acceptable excipient comprises a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof. Still further, in certain cases, the pharmaceutically acceptable excipient or pharmaceutically acceptable salt thereof comprises the carbohydrate or the pharmaceutically acceptable salt thereof, and wherein the carbohydrate or the pharmaceutically acceptable salt thereof comprises lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, a pharmaceutically acceptable salt of any of these, or any combination thereof. The lactose may comprise lactose milled lactose, sieved lactose, micronized lactose, spray dried lactose, at least substantially anhydrous lactose, monohydrate lactose, a pharmaceutically acceptable salt thereof, or any combination thereof. The powdery pharmaceutical composition described herein when stored in a sealed container placed in a room at 25° C. and a room atmosphere having about 50 percent relative humidity, may retain at least about: 90% of the agonist thereof after 6 months, as measured by HPLC.

In certain instances, regarding the agonist, the agonist is present in an amount ranging from about 0.001 mg to about 20 mg. In certain instances, the agonist can be in the form of a pharmaceutically acceptable salt. In comes cases the salt is a hydrochloride salt, a bitartrate salt or a borate salt.

In certain instances, the particles comprising the agonist comprise a median diameter of less than 5 μm. In certain instances, the particles comprising the agonist or the pharmaceutically acceptable salt thereof comprise a median diameter of less than about: 6 μm, 7 μm, 8 μm, 9 μm or 10 μm.

Certain other aspects concern a kit of an aforementioned pharmaceutical composition. The kit may include a packaging of at least a portion of the pharmaceutical formulation.

Certain other aspects of the disclosure concern a method of treating a disease or condition in a subject in need thereof, comprising treating the disease or condition by administering, via inhalation, a first therapeutic comprising a therapeutically effective amount of the powdery pharmaceutical composition above to the subject in need thereof. The method may comprise administering wherein the administering is conducted one, twice, three, or four times per day. The disease or condition may be selected from the group consisting of: a migraine headache, a tension headache and a cluster headache. The disease or condition may be from the group consisting of: a post-traumatic stress disorder, a depression, a generalized anxiety disorder, a panic disorder, a phobia, a social anxiety disorder, attention deficit hyperactivity disorder, an agoraphobia and a personality disorder.

In the method, the powdery pharmaceutical composition may be administered as needed, or for about: one day, two days, three days, four days, five days, six days, a week, two weeks, three weeks, a month, two months, three months, four months, five months, six months, seven months, eight months, nine months, ten months, eleven months, a year, or chronically. The method may include the administration of a second therapeutic or pharmaceutical salt thereof. The second therapeutic or pharmaceutical salt thereof can be administered concurrently or consecutively. In certain cases, when the first therapeutic is the substituted tryptamine, the second therapeutic comprises the N-Methyl-D-aspartate receptor agonist. The method may further include administering caffeine, a non-steroidal anti-inflammatory drug, an anti-calcitonin gene-related peptide monoclonal antibody or a combination thereof to the subject.

In certain instances, the subject is first diagnosed with a disease or condition. In some cases, the diagnosing comprises employing an in vitro diagnostic.

In certain instances, the method employs using the pharmaceutical wherein pharmaceutical composition is contained within a capsule, wherein the capsule is at least in part contained within an inhaler, and wherein the inhaler contains a sharp surface configured to puncture or slice the capsule, and wherein, prior to administrating, the inhaler is actuated such that the sharp surface punctures or slices the capsule. Administration may be via inhalation and may be an oral inhalation, intra nasal administration, or any combination thereof.

Other aspects of the disclosure pertain to a method for making a powdery pharmaceutical composition, comprising contacting in a solution: an agonist comprising a substituted tryptamine, or a pharmaceutically acceptable salt thereof, an amide of lysergic acid, or a pharmaceutically acceptable salt thereof, a N-Methyl-D-aspartate receptor agonist or a pharmaceutical acceptable salt thereof: a coating material comprising a fumaryl diketopiperazine (FDKP), 1,2-distearoyl-sn-glycero-3-phosphocholine, a hydroxypropyl methylcellulose (HPMC), a hydroxypropyl methylcellulose acetate succinate (HPMCAS), a povidone, a copovidone or any combination thereof, and ii) a solvent; wherein the solution is spray dried to form substantially encapsulated particles. In this formulation, the spray drying comprises: i) atomizing liquid droplets comprising the agonist or the pharmaceutically acceptable salt thereof, the coating material, and the solvent. ii) drying the droplets to form the substantially encapsulated particles, wherein the substantially encapsulated particles comprise the agonist or the pharmaceutically acceptable salt thereof substantially encapsulated by the coating material and iii) recovering the substantially encapsulated particles.

Other aspects of the disclosure pertain to a powdery pharmaceutical composition, comprising: i) particles of a pharmaceutically acceptable excipient; and ii) a plurality of spray dried particles, wherein each particle of the plurality of spray dried particles is substantially encapsulated in a coating material and wherein the plurality of spray dried particles substantially encapsulated in the coating material comprise N-lactoyl-phenylalanine or a pharmaceutically acceptable salt thereof; wherein within the plurality of spray dried particles substantially encapsulated in the coating material at least a portion of the spray dried particles substantially encapsulated in the coating material individually have a particle diameter ranging from about 1 micrometer to about 10 micrometers, as measured by a particle analyzer using laser diffraction; and wherein the coating material comprises a hydroxypropyl methylcellulose (HPMC), a fumaryl diketopiperazine (FDKP), 1,2-distearoyl-sn-glycero-3-phosphocholine, a hydroxypropyl methylcellulose acetate succinate (HPMCAS), a povidone, a copovidone or any combination thereof. In certain cases, this pharmaceutical formulation may be used for a method of treating a disease or condition in a subject in need thereof, comprising treating the disease or condition by administering, via inhalation, a first therapeutic comprising a therapeutically effective amount of the powdery pharmaceutical composition to the subject. The administration may be once, twice, three times, four times, or more per day. The administration may be daily. In certain cases, the disease or condition is selected from the group consisting of: obesity, increased appetite, or glucose intolerance

### EXAMPLES

The following examples are included for illustrative purposes only and are not intended to limit the scope of the disclosure.

### Example 1

The powdery pharmaceutical composition described herein is administered by a dry powder inhaler or by a nasal inhaled device. Referring to FIG. 1A, FIG. 1A shows a dry powder inhaler device for delivery of a powdery pharmaceutical composition described herein to the lung alveolar. The inhaler device comprises: a protective cap 101, a rotatable top comprising a mouthpiece 102, a lower base chamber receptacle for placing the pharmaceutical capsule 103, lateral buttons for mechanically piercing the capsule with a sharp surface while inside the chamber 104, wherein the chamber is aerially connected to the mouthpiece permitting inhalation of capsule contents. FIG. 1B shows the nasal administration by a nasal inhaled device of a powdery pharmaceutical composition in a human subject. The composition is inhaled via the nares after the capsule containing the composition is pierced within the nasal inhaled device.

### Example 2

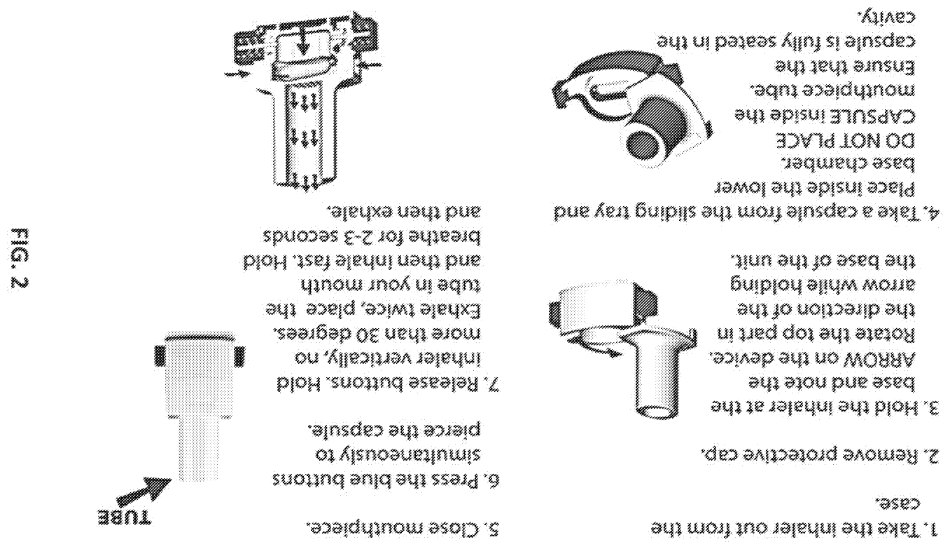

The method of using an inhaler device for the administration of a dry powdery pharmaceutical composition is shown in FIG. 2. The process for administration of the dry powdery pharmaceutical composition comprises 7 steps. Step 1: The inhaler is removed from the case. Step 2: The protective cap is removed. Step 3: The inhaler is held at the base and the top part is rotated in the direction of the arrow while the base of the unit is held. Step 4: A capsule is placed inside the lower base chamber cavity. Step 5: The mouthpiece is closed. Step 6: The buttons are pressed simultaneously to piece the capsule. Step 7: The buttons are released. The inhaler is held vertically, e.g., no more that about 30 degrees. The subject exhales twice before placing the tube in their mouth. The subject inhales quickly and holds their breath for about 2-3 seconds before exhaling.

### Example 3

The active ingredients (e.g., a serotonin receptor agonist) in a dry powdery pharmaceutical composition described herein is manufactured by a spray drying system. FIG. 3 shows a spray drying manufacturing system comprising a closed spray dryer container which receives the solution comprising a drug dissolved or mixed in a suitable solvent (aqueous or solvent based). The solution then enters the particle formation chamber which is connected to an atomizer located at the top of the chamber. The atomizer is a two component or rotary nozzle type that distributes the solution into fine droplets controlled by the atomizer pressure. This atomization gas is an inert gas, such as air, nitrogen or carbon dioxide. The atomized droplets go through a hot gas drying chamber to produce uniform fine particles that maintain a tight particle size distribution following liquid evaporation. Solid particle form and fall to the bottom of the drying chamber. The balance between temperature, flow rate, and droplet size, controls the drying process. The powder is recovered from the exhaust gas using a cyclone or a bag filter. Particle size is validated by a Malvern particle analyzer prior to blending with an excipient carrier. The active powder is blended with an excipient carrier (lactose) product in a Patterson Kelly (PK Blender) and the blended powder is fed to a hopper. From the hopper, the dry powder is placed into a Size 3 Hypromellose capsule, by a Bosch Encapsulator machine.

### Example 4

A male subject perceives an aura which indicates of the onset of a migraine. An aura usually occurs within an hour before head pain begins and generally lasts less than 60 minutes. Alternatively, the male subject does not feel pain after the aura but suffers loss of direct vision while peripheral vision still functions. The subject is administered a pharmaceutical composition to treat the upcoming migraine headache. The pharmaceutical composition comprises an encapsulated serotonin agonist which has been processed to an encapsulated dry powder using the methods described herein (e.g., spay drying). The dry powder is mixed with a lactose powder. The serotonin agonist is packaged in a capsule and is administered with an inhaler. The dosing regimen comprises an effective amount of a serotonin agonist to treat the allergic reaction. The absorption of the inhaled pharmaceutical composition reaches the blood stream at least 5× faster than a comparable pharmaceutical composition that is administered orally.

### Example 5

The powdery pharmaceutical composition described herein is administered by a dry powder inhaler. Referring to FIG. 9, FIG. 9 shows a dry powder inhaler device for delivery of a powdery pharmaceutical composition described herein to the lung alveolar. The inhaler device comprises: a protective cap 201, a rotatable top comprising a mouthpiece 202, a lower base chamber receptacle 206 for placing the pharmaceutical capsule 203, lateral buttons for mechanically piercing the capsule with a sharp surface 204 while inside the chamber with the use of a spring 205, wherein the chamber is aerially connected to the mouthpiece permitting inhalation of capsule contents. The baseplate 207 is fitted to the lower base chamber receptacle.

### Example 6

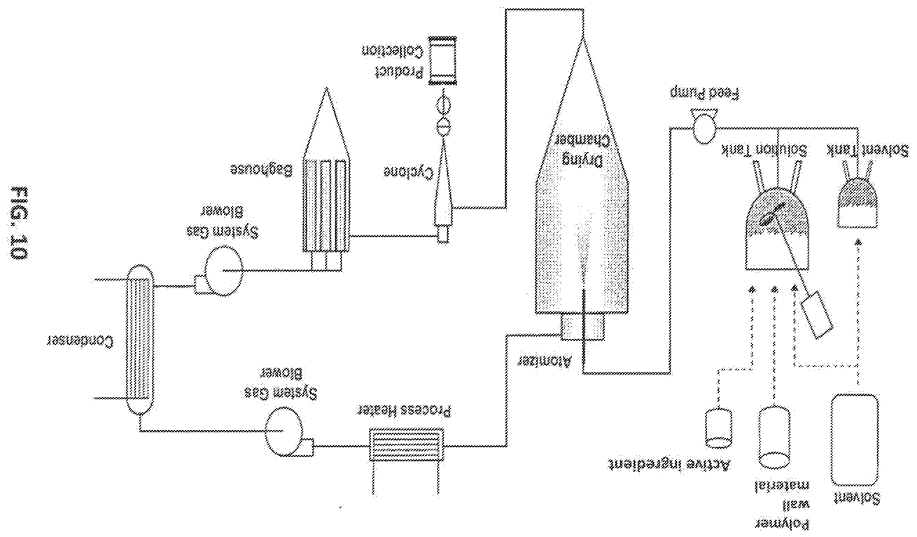

The active encapsulated ingredient (e.g., psilocybin) in a dry powdery pharmaceutical composition described herein was manufactured by a spray drying system. FIG. 10 shows a spray drying manufacturing system comprising a closed spray drying chamber which receives the solution comprising a polymer wall material (e.g., HPMCAS) dissolved in a solvent (e.g., 70% ethanol and 30% water) and an active ingredient (e.g., psilocybin or a pharmaceutically acceptable salt thereof). The dissolved polymer wall material and psilocybin were thoroughly mixed into a liquid suspension. The liquid suspension was then fed into an atomizer located at the top of the chamber. The atomizer is a two component or rotary nozzle type that distributes the solution into fine droplets controlled by the atomizer pressure. This atomization gas is an inert gas, such as air, nitrogen or carbon dioxide. The atomized droplets were then sent through a drying chamber with hot gas to produce uniform fine particles that maintain a tight particle size distribution following liquid evaporation. Solid particles were formed and fell to the bottom of the drying chamber as amorphous crystals. The balance between temperature, flow rate, and droplet size, were used to control the drying process. The powder was recovered from the exhaust gas using a cyclone or a bag filter. Particle size was validated by a Malvern particle analyzer prior to blending with an excipient carrier.

The active powder was blended with an excipient carrier (lactose) product in a Patterson Kelly (PK Blender) and the blended powder was fed to a hopper. From the hopper, the dry powder was placed into a Size 3 Hypromellose capsule, by a Bosch Encapsulator machine. Small amounts of microencapsulated psilocybin or a pharmaceutically acceptable salt thereof was blended with a V-type blender using an intensifier bar that operated at high speeds to distribute the active powder uniformly into the excipient carrier. The V-Blenders are manufactured by Patterson Kelly/PK Blender, Gemco or Ross blenders.

The blended powder was then loaded into the hopper of the encapsulator machine (“encapsulator”), which fed the powder into the capsules. The encapsulator automatically separated the capsule top (“cap”) and body(“shell”) and the powder was slugged and then transferred into the body of the capsule. The capsule halves were closed together to form an enclosed capsule that contained the blended powder. During filling, the capsule atmosphere was made inert with nitrogen to prevent oxidation and remove moisture from the blend so that the inhalable powder was able flow freely from the capsule using the dry powder inhaler. The dry powder was placed into a Hypromellose capsule, by a Bosch, ACG or IMA Encapsulator machine.

### Example 7

A male subject has migraine headaches. The subject is administered a pharmaceutical composition to treat the migraine headaches. The pharmaceutical composition comprises an encapsulated serotonin agonist (e.g., psilocin) which has been processed to an encapsulated dry powder using the methods described herein (e.g., spay drying). The dry powder is mixed with a lactose powder. The serotonin agonist is packaged in a capsule and is administered intranasally with a device. The dosing regimen comprises an effective amount of the serotonin agonist to treat the headache. The absorption of the nasally inhaled pharmaceutical composition reaches the blood stream at least 5× faster than a comparable pharmaceutical composition that is administered orally.

### Example 8

A male subject is diagnosed with obesity. The subject is administered a pharmaceutical composition to treat obesity. The pharmaceutical composition comprises an encapsulated tryptamine in combination with N-lactoyl-phenylalanine which have been processed to an encapsulated dry powder using the methods described herein (e.g., spay drying). The dry powder is mixed with a lactose powder. The N-lactoyl-phenylalanine and the triptan are packaged in a capsule and are administered with an inhaler. The dosing regimen comprises an effective amount of triptan and N-lactoyl-phenylalanine to treat the obesity. Effective treatment is determined by measuring the subject's weight prior to the first administration of the treatment as compared to the subject's weight after administration of the treatment for a specified time (e.g., one month). Administration continues daily, or weekly until a targeted decrease in weight of the subject is achieved. The absorption of the inhaled pharmaceutical composition reaches the blood stream at least 5× faster than a comparable pharmaceutical composition that is administered orally.

### Example 9

A male subject is diagnosed with an opioid addiction. The subject is administered a pharmaceutical composition to block the craving for the opioid. The pharmaceutical composition comprises encapsulated ibogaine processed to an encapsulated dry powder using the methods described herein (e.g., spay drying). The dry powder is mixed with an excipient (e.g., lactose) and encapsulated. Ibogaine is packaged in a capsule and is administered with an inhaler. The dosing regimen comprises an effective amount ibogaine to treat the opioid addiction as measured by the subject's self-assessment of opioid craving. Administration continues daily, or weekly until opioid craving is eliminated. The absorption of the inhaled pharmaceutical composition reaches the blood stream at least 5× faster than a comparable pharmaceutical composition that is administered orally.

### Example 10

Three male subjects and one female subject were administered doses of 5 mg, 10 mg, and 15 mg of psilocybin. Each individual dosage level was administered about three weeks apart for the treatment of generalized anxiety and/or post-traumatic stress disorder (PTSD). In a separate experiment, the subjects were administered doses of 1 mg, 5 mg, and 10 mg of 4-AoC-DMT (O-Acetylpsilocin). Each individual dosage level was administered about three weeks apart for the treatment of generalized anxiety and/or post-traumatic stress disorder (PTSD). The dry powder pharmaceutical compositions comprised psilocybin or 4-AoC-DMT encapsulated by HPMCAS using the methods described herein (e.g., spay drying). The encapsulated dry powder was administered with an inhaler. No adverse effects were noted besides 10 mg of 4-AoC-DMT being noted as “too much” as indicated by 3 male patients and 1 female patient. Overall, the doses of 5 mg, 10 mg and 15 mg of psilocybin and the doses of 1 mg, and 5 mg of 4-AoC-DMT reduced anxiety in the subjects.

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the methods presented in the disclosure. It is intended that the following claims define the scope of the disclosure and that methods and structures within the scope of these claims and their equivalents be covered thereby.

What is claimed is:

1. A powdery pharmaceutical composition, comprising:
a) particles of a pharmaceutically acceptable excipient; and
b) a plurality of spray dried particles, wherein each particle of the plurality of spray dried particles is substantially encapsulated in a coating material and wherein the plurality of spray dried particles substantially encapsulated in the coating material comprise:
an agonist selected from the group consisting of: a substituted tryptamine or a pharmaceutically acceptable salt thereof, an amide of lysergic acid or a pharmaceutical acceptable salt thereof, and a N-methyl-d-aspartate receptor agonist or a pharmaceutical acceptable salt thereof; and
wherein the spray dried particles substantially encapsulated in the coating material individually have a particle diameter ranging from about 1 micrometer to about 10 micrometers, as measured by a particle analyzer using laser diffraction; and
wherein the coating material comprises a trehalose, a hydroxypropyl methylcellulose (HPMC), a fumaryl diketopiperazine (FDKP), a 1,2-distearoyl-sn-glycero-3-phosphocholine, a hydroxypropyl methylcellulose acetate succinate (HPMCAS), a povidone, a copovidone, or any combination thereof.

2. The powdery pharmaceutical composition of claim 1, wherein the spray dried particles comprise the substituted tryptamine or the pharmaceutically acceptable salt thereof.

3. The powdery pharmaceutical composition of claims 2, wherein the substituted tryptamine is a psilocin or a salt thereof.

4. The powdery pharmaceutical composition of claim 2, wherein the substituted tryptamine is a psilocybin or a salt thereof.

5. The powdery pharmaceutical composition of claim 2, wherein the substituted tryptamine is an ibogaine or a salt thereof.

6. The powdery pharmaceutical composition of claim 1, wherein the spray dried particles comprise the N-Methyl-D-aspartate receptor agonist or the pharmaceutically acceptable salt thereof.

7. The powdery pharmaceutical composition of claim 6, wherein the N-Methyl-D-aspartate receptor agonist is a ketamine or a salt thereof.

8. The powdery pharmaceutical composition of claim 1, wherein the powdery pharmaceutical composition is for inhaled use or for intranasal use. The powdery pharmaceutical composition of claim 1, wherein the pharmaceutical composition is in unit dose form.

10. The powdery pharmaceutical composition of claim 1, wherein at least a portion of the particles of the pharmaceutically acceptable excipient individually have a particle diameter ranging from about 30 micrometers to about 100 micrometers.

11. The powdery pharmaceutical composition of claim 1, wherein the particles of the pharmaceutically acceptable excipient and the plurality of spray dried particles are admixed into a substantially homologous mixture.

12. The powdery pharmaceutical composition of claim 1, which is contained within a capsule.

13. The powdery pharmaceutical composition of claim 12, wherein the capsule is about one quarter to about one half, by volume, filled with the powdery pharmaceutical composition.

14. The powdery pharmaceutical composition of claim 13, wherein a weight-to-weight ratio of: a) the particles of the pharmaceutically acceptable excipient and b) the plurality of spray dried particles, ranges from about 1:1 (w/w) to about 10000:1 (w/w).

15. The powdery pharmaceutical composition of claim 14, wherein the weight-to-weight ratio of: a) the particles of the pharmaceutically acceptable excipient and b) the plurality of spray dried particles, ranges from about 1:1 (w/w) to about 10:1 (w/w).

16. The powdery pharmaceutical composition of claim 12, wherein the portion of the capsule not containing the powdery pharmaceutical composition comprises an inert gas.

17. The powdery pharmaceutical composition of claim 12, wherein the capsule comprises a hydroxypropylmethyl cellulose (HPMC) capsule.

18. The powdery pharmaceutical composition of claim 12, wherein the capsule is size: 000, 00, 0, 1, 2, 3, or 4.

19. The powdery pharmaceutical composition of claim 18, comprising the capsule, wherein the capsule is size 3.

20. The powdery pharmaceutical composition of claim 1, contained within an inhaler unit.

21. The powdery pharmaceutical composition of claim 12, wherein the capsule is contained in an inhaler unit.

22. The powdery pharmaceutical composition of claim 1, wherein the pharmaceutically acceptable excipient comprises a carbohydrate, an alginate, a povidone, a carbomer, a flavor, a natural gum, a silicone, an alcohol, a butter, a wax, a fatty acid, a preservative, a pharmaceutically acceptable salt of any of these, or any combination thereof.

23. The powdery pharmaceutical composition of claim 22, wherein the pharmaceutically acceptable excipient or pharmaceutically acceptable salt thereof comprises the carbohydrate or the pharmaceutically acceptable salt thereof, and wherein the carbohydrate or the pharmaceutically acceptable salt thereof comprises a lactose, a microcrystalline cellulose, a cellulose, a mannitol, a sorbitol, a starch, a starch glycolate, a hydroxypropyl methylcellulose, a hydroxypropyl methylcellulose acetate succinate, a cyclodextrin, a maltodextrin, a croscarmellose sodium, a corn starch, a carrageenan, a sorbitol, a maltitol, a glucose, a pharmaceutically acceptable salt of any of these, or any combination thereof.

24. The powdery pharmaceutical composition of claim 23, wherein the pharmaceutically acceptable excipient or the pharmaceutically acceptable salt thereof comprises the lactose or the pharmaceutically acceptable salt thereof.

25. The powdery pharmaceutical composition of claim 24, comprising the lactose or the pharmaceutically acceptable salt thereof, which comprises a milled lactose, a sieved lactose, a micronized lactose, a spray dried lactose, an at least substantially anhydrous lactose, a monohy drate lactose, a pharmaceutically acceptable salt thereof, or any combination thereof.

26. The powdery pharmaceutical composition of claim 1, wherein the powdery pharmaceutical composition retains at least about 90% of the agonist after about 6 months when stored in a sealed container at 25° C. and a room atmosphere having about 50 percent relative humidity, as measured by high-performance liquid chromatography (HPLC).

27. The powdery pharmaceutical composition of claim 1, wherein the agonist is present in an amount ranging from about 0.001 mg to about 20 mg.

28. The powdery pharmaceutical composition of claim 1, wherein the agonist is in the form of a pharmaceutically acceptable salt thereof and is a hydrochloride salt, a bitartrate salt, or a borate salt.

29. The powdery pharmaceutical composition of claim 1, wherein the particles comprising the agonist comprise a median diameter of less than 5 μm.

30. The powdery pharmaceutical composition of claim 1, wherein the particles comprising the agonist or the pharmaceutically acceptable salt thereof comprise a median diameter of less than about: 6 μm, 7 μm, 8 μm, 9 μm or 10 μm.

31. A kit comprising the powdery pharmaceutical composition of claim 1 contained at least in part in a container.

32. A method of treating a disease or condition in a subject in need thereof, comprising administering by inhalation administration, a first therapeutic comprising a therapeutically effective amount of the powdery pharmaceutical composition of claim 1 to the subject in need thereof thereby treating the disease or condition.

33. The method of claim 32, wherein the administering is conducted one, twice, three, or four times per day.

34. The method of claim 32, wherein the disease or condition is selected from the group consisting of: a migraine headache, a tension headache and a cluster headache.

35. The method of claim 32, wherein the disease or condition is selected from the group consisting of: a post-traumatic stress disorder, a depression, a generalized anxiety disorder, a panic disorder, a phobia, a social anxiety disorder, an attention deficit hyperactivity disorder, an agoraphobia and a personality disorder.

36. The method of claim 32, wherein the disease or condition is selected from the group consisting of: an alcohol addiction, a cocaine addiction, a methamphetamine addiction, an opioid addiction, a nicotine addiction, and a gambling addiction.

37. The method of claim 32, wherein the powdery pharmaceutical composition is administered as needed, or for about: one day, two days, three days, four days, five days, six days, a week, two weeks, three weeks, a month, two months, three months, four months, five months, six months, seven months, eight months, nine months, ten months, eleven months, a year, or chronically.

38. The method of claim 32, wherein a second therapeutic or pharmaceutically acceptable salt thereof is administered.

39. The method of claim 38, wherein the second therapeutic or a pharmaceutically acceptable salt thereof is administered concurrently or consecutively.

40. The method of claim 38, wherein the second therapeutic or the pharmaceutically acceptable salt thereof is comprised in the powdery pharmaceutical formulation.

41. The method of claim 38, wherein the first therapeutic comprises the substituted tryptamine or the pharmaceutically acceptable salt thereof and the second therapeutic comprises the N-Methyl-D-aspartate receptor agonist or the pharmaceutically acceptable salt thereof.

42. The method of claim 38, wherein the first therapeutic comprises the amide of lysergic acid or the pharmaceutically acceptable salt thereof and the second therapeutic comprises the N-Methyl-D-aspartate receptor agonist or the pharmaceutically acceptable salt thereof.

43. The method of claim 32, further comprising administering a caffeine, a non-steroidal anti-inflammatory drug, an anti-calcitonin gene-related peptide monoclonal antibody or a combination thereof to the subject.

44. The method of claim 32, wherein the subject is diagnosed with the disease or condition.

45. The method of claim 44, wherein the diagnosing comprises employing an in vitro diagnostic.

46. The method of claim 45, wherein the in vitro diagnostic is a companion diagnostic.

47. The method of claim 32, wherein the powdery pharmaceutical composition is contained within a capsule, wherein the capsule is at least in part contained within an inhaler, and wherein the inhaler contains a sharp surface configured to puncture or slice the capsule, and wherein, prior to administrating, the inhaler is actuated such that the sharp surface punctures or slices the capsule.

48. The method of claim 32, wherein the inhalation administration is oral inhalation, intra nasal administration, or any combination thereof.

49. A powdery pharmaceutical composition, comprising:
a) particles of a pharmaceutically acceptable excipient; and
b) a plurality of spray dried particles, wherein each particle of the plurality of spray dried particles is substantially encapsulated in a coating material and wherein the plurality of spray dried particles substantially encapsulated in the coating material comprise a N-lactoyl-phenylalanine or a pharmaceutically acceptable salt thereof;
wherein at least a portion of the spray dried particles substantially encapsulated in the coating material individually have a particle diameter ranging from about 1 micrometer to about 10 micrometers, as measured by a particle analyzer using laser diffraction; and wherein the coating material comprises trehalose, a hydroxypropyl methylcellulose (HPMC), a fumaryl diketopiperazine (FDKP), 1,2-distearoyl-sn-glycero-3-phosphocholine, a hydroxypropyl methylcellulose acetate succinate (HPMCAS), a povidone, a copovidone or any combination thereof.

50. A method of treating a disease or condition in a subject in need thereof, comprising administering by inhalation administration, a composition comprising a therapeutically effective amount of the powdery pharmaceutical composition of claim 49 to the subject in need thereof thereby treating the disease or condition.

51. The method of claim 50, wherein the administering is conducted one, twice, three, or four times per day.

52. The method of claim 50, wherein the disease or condition is selected from the group consisting of: an obesity, an increased appetite, or a glucose intolerance.
